(19) United States
(12) Patent Application Publication
(10) Pub. No.: US 2020/0124592 A1
(43) Pub. Date: Apr. 23, 2020

(54) MAGNETIC NANOPARTICLE

(71) Applicant: APOSTLE, INC., Sunnyvale, CA (US)

(72) Inventors: Bo ZHANG, Palo Alto, CA (US); Dongliang GE, Redwood City, CA (US); Xin GUO, Palo Alto, CA (US)

(21) Appl. No.: 16/627,212

(22) PCT Filed: Jun. 28, 2018
(86) PCT No.: PCT/US2018/040277

§ 371 Date: Dec. 27, 2019

Related U.S. Application Data
(60) Provisional application No. 62/527,695, filed on Jun. 30, 2017.

(51) Int. Cl. G01N 33/543; G01N 1/28; C12N 15/10; G01N 33/574; C12Q 1/6806

(52) U.S. Cl. CPC G01N 33/5434; G01N 1/28; C12Q 1/6806; G01N 33/574; C12N 15/1013

(57) ABSTRACT

Disclosed herein are magnetic nanoparticles, compositions and kits comprising the magnetic nanoparticles, methods of making the magnetic nanoparticles, and methods of using the magnetic nanoparticles to enrich biological targets.

### CROSS-REFERENCE TO RELATED APPLICATIONS

This application claims the benefit of U.S. Provisional Application Ser. No. 62/527,695 filed Jun. 30, 2017, which is incorporated in its entirety herein by this reference.

### BACKGROUND

Magnetically separable particles are commonly used for enriching biological molecules, such as nucleic acids, proteins, peptides, carbohydrates, lipids, as well as cells and cellular organelles. Magnetic particles larger than 1 μm in diameter are generally more magnetically responsive. But they have a limited surface area per unit mass and a high sedimentation rate, which restrict their interaction with target materials. Smaller magnetic beads have a larger surface area per unit mass and a lower sedimentation rate, allowing sufficient interaction with target materials. But they are less magnetically responsive and harder to separate with external magnetic force.

Magnetic particles that are presently in wide use are 1-10 μm in diameter and are composed of polymer, such as polystyrene, containing dispersed small (<30 nm) magnetic nanoparticles. In additional to the disadvantages caused by size, the benzene groups of polymer interact with nucleic acids through 7C-7C stacking. Nucleic acids with larger size are therefore less likely to be released from such magnetic beads during the elution step compared to the smaller nucleic acids, due to 7C-7C stacking, leading to a bias towards the smaller size nucleic acids in the isolated sample.

Silica material can bind to nucleic acids in the presence of high concentration chaotropic agents and can release the bound nucleic acids in the absence of chaotropic agents. Spin columns containing a silica membrane that can selectively bind to DNA or RNA are commonly used in laboratories. Purification with silica membrane delivers high-purity nucleic acids suitable for many downstream applications. However, the process involves multiple centrifugation steps, which makes it time-consuming and challenging for automation. Compared to magnetic particles, the limited interaction between the membrane of spin columns and the target molecules results in a less efficient DNA or RNA purification.

There is, therefore, a need for new magnetic particles with combined advantages of low sedimentation rate, adequate surface area per unit mass, strong magnetic response, and reduced nucleic acid size bias. The magnetic nanoparticle should allow efficient purification and be suitable for automation during high throughput processes.

### SUMMARY

We have designed and produced single core magnetic nanoparticles with a size range of 100-1000 nm in diameter. These magnetic nanoparticles have low sedimentation rate, adequate surface area per unit mass, strong magnetic response, and optimized surface chemistry, and can be easily adapted to automated processes.

Accordingly, in a first aspect, disclosed herein is a magnetic nanoparticle comprising a single magnetic core and an outer shell, wherein the outer shell covers the magnetic core. In some embodiments, the magnetic particle has a maximum diameter of 100 nm to 1000 nm. In some embodiments, the magnetic particle has a maximum diameter of 300 nm to 700 nm. In some embodiments, the magnetic particle has a maximum diameter of 400 nm to 600 nm.

In various embodiments, the magnetic core is composed of metal oxide. In some embodiments, the metal oxide is an iron oxide. In certain embodiments, the iron oxide is Fe₃O₄. In certain embodiments, the metal oxide is XFe₂O₄, wherein X is selected from the group consisting of Mn, Ca, Co, Zn, Cu, Mg, Ba, Ni, and Cr. In some embodiments, the magnetic core has a diameter of 100 nm to 800 nm. In some embodiments, the magnetic core has a diameter of 200 nm to 600 nm. In some embodiments, the magnetic core has a diameter of 300 nm to 400 nm.

In some embodiments, the outer shell comprises silicon dioxide or titanium dioxide. In certain embodiments, the silicon dioxide or titanium dioxide is amorphous. In certain other embodiments, the silicon dioxide or titanium dioxide is in crystallized form.

In some embodiments, the outer shell comprises polymer. In some of these embodiments, the polymer is selected from the group consisting of: polyethylene glycol, polyacrylic acid, polyacrylamide, polyvinyl alcohol, poly-methyl methacrylate, polystyrene, poly-4-vinylphenol, polyester, polyimide, polyethylene, polypropylene, polyethylene vinyl acetate, polyacrylates, and polysaccharide.

In some embodiments, the outer shell comprises mesoporous structure. In some embodiments, the mesoporous structure has an average surface pore diameter of 1 nm to 30 nm. In some embodiments, the mesoporous structure has an average surface pore diameter of 1 nm to 10 nm. In some embodiments, the mesoporous structure has an average surface pore diameter of 10 nm to 20 nm. In some embodiments, the mesoporous structure has an average surface pore diameter of 20 nm to 30 nm.

In some embodiments, the magnetic nanoparticle further comprises a functional group on the surface of the outer shell. In various embodiments, the functional group is selected from the group consisting of: carboxyl, hydroxyl, epoxy, carbonyl, aldehyde, amine, maleimide, N-hydroxysuccinimide, carbodiimide, anhydride, hydrazide, biotin, and polyethylene glycol.

In some embodiments, the magnetic nanoparticle further comprises a polynucleotide, a polysaccharide, a polypeptide, a protein, an aptamer, or an ion. In certain embodiments, the polynucleotide has a length of 10 to 100 bases. In certain embodiments, the polynucleotide hybridizes specifically to a DNA or an RNA target. In specific embodiments, the polynucleotide is polydT. In certain embodiments, the polynucleotide binds to a protein target. In certain embodiments, the protein is an antibody. In certain embodiments, the protein is Protein A, Protein G, Protein A/G, or Protein L. In certain embodiments, the protein is streptavidin, avidin, or NeutrAvidin.

In some embodiments, the magnetic nanoparticle has a positive surface charge. In some embodiments, the magnetic nanoparticle has a negative surface charge. In certain embodiments, the surface charge of the magnetic nanoparticle changes according to pH of a solution.

In another aspect, provided herein is a composition comprising a plurality of magnetic nanoparticles and optionally an aqueous solution. In some embodiments, at least 40% of the magnetic particles have the same maximum diameter. In some embodiments, at least 60% of the magnetic particles have the same maximum diameter. In some embodiments, at least 80% of the magnetic particles have the same maximum diameter.

In another aspect, provided herein is a kit for isolating a biological target comprising the composition and optionally a buffer or a combination of buffers. In certain embodiments, the kit further comprises a chaotropic agent.

In another aspect, disclosed herein is a method of enriching one or more biological target from a biological medium, comprising: a) providing a sample of biological medium containing one or more biological target; b) adding to the sample the composition comprising dispersed magnetic nanoparticles capable of binding the biological target, under conditions that permit a complex to form between the magnetic nanoparticle and the biological target; c) separating the complex from the biological medium by application of an external magnetic field; and d) recovering the biological target from the magnetic nanoparticles. In some embodiments, the method further comprises pretreating the sample of biological medium to effect the release of the biological target. In some embodiments, the method further comprises pretreating the sample of biological medium to remove contaminants. In some embodiments, the method further comprises contacting the sample with a molecular probe, the molecular probe comprising a moiety with high affinity for a molecule on the magnetic nanoparticle, wherein the molecular probe binds specifically to the biological target.

In various embodiments, the biological target is selected from the group consisting of a nucleic acid, a peptide, a protein, a carbohydrate, a lipid, a cell, and an exosome. In certain embodiments, the biological target is a nucleic acid. In some of these embodiments, the nucleic acid is circulating free DNA (cfDNA) or circulating free RNA (cfRNA). In certain embodiments, the biological target is a cell. In some of these embodiments, the cell is a circulating tumor cell (CTC). In certain embodiments, the biological medium is a body fluid. In some of these embodiments, the body fluid is blood, serum, plasma, saliva, cerebrospinal fluid, urine, semen, or ascites.

In another aspect, disclosed herein is a method of enriching circulating free DNA (cfDNA) from a body fluid, comprising: a) providing a sample of body fluid containing the cfDNA; b) adding to the sample a solution of dispersed magnetic nanoparticles capable of binding the cfDNA, under conditions that permit a complex to form between the magnetic nanoparticle and the cfDNA; c) separating the complex from the body fluid by application of an external magnetic field; and d) recovering the cfDNA from the magnetic nanoparticles. In some embodiments, a) further comprises pretreating the body fluid to remove cells. In some embodiments, a) further comprises pretreating the body fluid to remove proteins. In some embodiments, c) further comprises washing the complex to remove contaminants. In some embodiments, the method further comprises e) sequencing the entirety or a portion of the enriched cfDNA.

In some embodiments, the cfDNA is less than 100 bp. In certain embodiments, the cfDNA is a single-stranded DNA (ssDNA).

In certain embodiments, the body fluid is from a patient with, or suspected of having, cancer. In certain embodiments, the body fluid is from a patient with, or suspected of having, an infectious disease. In certain embodiments, the body fluid is from a pregnant woman.

In another aspect, disclosed herein is a method of enriching circulating free RNA (cfRNA) from a body fluid, comprising: a) providing a sample of body fluid containing the cfRNA; b) adding to the sample a solution of dispersed magnetic nanoparticles capable of binding the cfRNA, under conditions that permit a complex to form between the magnetic nanoparticle and the cfRNA; c) separating the complex from the body fluid by application of an external magnetic field; and d) recovering the cfRNA from the magnetic nanoparticles. In some embodiments, a) further comprises: pretreating the body fluid to remove cells. In some embodiments, a) further comprises pretreating the body fluid to remove proteins. In some embodiments, c) further comprises: washing the complex to remove contaminants. In some embodiments, the method further comprises e) sequencing the entirety or a portion of the enriched cfRNA.

In some embodiments, the cfRNA is less than 100 nt. In certain embodiments, the cfRNA is a miRNA.

In certain embodiments, the body fluid is from a patient with, or suspected of having, cancer. In certain embodiments, the body fluid is from a patient with, or suspected of having, an infectious disease. In certain embodiments, the body fluid is from a pregnant woman.

In another aspect, disclosed herein is a method of enriching circulating tumor cell (CTC) from a body fluid, comprising: a) providing a sample of body fluid containing the circulating tumor cell; b) adding to the sample a solution of dispersed magnetic nanoparticles capable of binding the circulating tumor cell, under conditions that permit a complex to form between the magnetic nanoparticle and the circulating tumor cell; and c) separating the complex from the body fluid by application of an external magnetic field. In some embodiments, a) further comprises: pretreating the body fluid to enrich cells. In some embodiments, c) further comprises: washing the complex to remove contaminants. In some embodiments, the method further comprises: recovering the circulating tumor cell from the magnetic nanoparticles. In some embodiments, the method further comprises: analyzing the circulating tumor cell. In some of these embodiments, the analyzing the circulating tumor cell is analyzing the size and shape of the circulating tumor cell, analyzing the surface biomarker of the circulating tumor cell, or sequencing the DNA/RNA of the circulating tumor cell.

In certain embodiments, the body fluid is from a patient with, or suspected of having, cancer.

In another aspect, provided herein is a method of preparing a magnetic nanoparticle, comprising: a) making a dispersion comprising a metal salt, an organic solvent, and a capping reagent; b) heating the dispersion; c) isolating magnetic cores from the dispersion; d) adding a silicon or titanium organic compound to the magnetic cores; e) hydrolyzing at least some of the silicon or titanium organic compound; and f) crosslinking the hydrolyzed silicon or titanium organic compound on the surface of the magnetic cores.

In some embodiments, the metal salt is an iron salt. In some embodiments, the metal salt is an iron salt and a salt of a second metal. In some of these embodiments, wherein the second metal is selected from the group consisting of Mn, Ca, Co, Zn, Cu, Mg, Ba, Ni, and Cr. In certain embodiments, the heating comprises heating the dispersion to 180-240° C. for 4-80 hours. In certain embodiments, the isolating comprises cooling, washing, and drying. In certain embodiments, the dispersion further comprises a first surfactant.

In various embodiments, before adding the silicon or titanium organic compound to the magnetic cores, d) further comprises dispersing the magnetic cores in solution comprising a second surfactant, wherein the second surfactant self-assembles on the magnetic core. In some of these embodiments, the method further comprises g) removing the self-assembled surfactant by ion exchange. In some embodiments, the magnetic nanoparticle is mesoporous.

### BRIEF DESCRIPTION OF THE DRAWINGS

These and other features, aspects, and advantages of the present invention will become better understood with regard to the following description, and accompanying drawings, where:

FIGS. 1A, 1B, 1C, and 1D show images of the magnetic nanoparticles described herein, with FIG. 1A being a digital photograph of the magnetic nanoparticles dispersed in a solution; and FIGS. 1B, 1C, and 1D showing scanning electron microscopy images of the magnetic nanoparticles.

FIG. 2 shows the structure of the magnetic core by scanning electron microscopy of a sample dried on silicon wafer.

FIGS. 3A and 3B are size distribution graphs of various magnetically responsive particle preparations, with FIG. 3A showing the size distribution of the magnetic nanoparticle described herein (“Apostle Minimax magnetic particle”); and FIG. 3B showing the size distribution of magnetic beads from other suppliers. The particle size of the magnetic nanoparticle was measured by dynamic light scattering particle size analyzer.

FIG. 4 shows the purification result of a DNA ladder sample (50-3000 bp) spiked in TE buffer and isolated by contact with and elution from the magnetic nanoparticles described herein (solid line) and a commercially available magnetic bead (dotted line). The DNA samples were analyzed by an Agilent 2100 bioanalyzer.

FIGS. 5A and 5B show the purification result of a DNA ladder sample (50-3000 bp) spiked in fetal bovine serum (FBS) and isolated by contact with and elution from magnetically responsive particles, with FIG. 5A showing the purification result by the magnetic nanoparticles described herein (solid line) and a commercially available magnetic bead (dotted line); and FIG. 5B showing a comparison of the DNA ladder enriched by contact with and elution from the magnetic nanoparticles described herein (solid line) with the original DNA ladder (dotted line). The DNA samples were analyzed by an Agilent 2100 bioanalyzer.

FIG. 6 represents the purification result of DNA from human plasma isolated by contact with and elution from the magnetic nanoparticles described herein (solid line) and by a commercially available magnetic bead (dotted line). The DNA samples were analyzed by an Agilent 2100 bioanalyzer.

FIG. 7 represents the purification result of DNA from human urine isolated by contact with and elution from the magnetic nanoparticles described herein (solid line) and by a commercially available magnetic bead (dotted line). The DNA samples were analyzed by an Agilent 2100 bioanalyzer.

FIG. 8 shows the purification result of a DNA ladder sample spiked in serum by three different batches of the magnetic nanoparticles described herein.

FIGS. 9A and 9B show the qPCR result of a DNA fragment containing the EGFR c.2573T>G (L858R) mutation (synthetic, ˜170 bp). The DNA fragment was isolated from TE buffer or serum by the magnetic nanoparticles described herein. FIG. 9A shows the amplification plot of the isolated DNA fragment and the original DNA fragment. FIG. 9B shows the qPCR standard curve of the isolated DNA fragment and the original DNA fragment.

FIG. 10 represents the purification result of a DNA ladder sample (50-3000 bp) spiked in serum isolated by contact with and elution from the magnetic nanoparticles described herein (solid line) as compared to the original DNA ladder (dotted line). The DNA samples were analyzed by an Agilent 2100 bioanalyzer. The arrow highlights DNA fragments of ˜50 bp.

FIGS. 11A, 11B, and 11C represents the purification result of cfDNA Reference Standard spiked in TE buffer by contact with and elution from the magnetic nanoparticles, with FIG. 11A representing the purification result by the magnetic nanoparticles described herein (solid line) compared to the original cfDNA Reference Standard (dotted line); FIG. 11B representing the purification result by the magnetic nanoparticles described herein (solid line) and by a commercially available magnetic bead (dotted line); and FIG. 11C representing a zoom in of FIG. 11B in the region between 35 bp and 100 bp. The arrows in FIG. 11B highlight DNA fragments of between 35 bp and 200 bp. The arrow in FIG. 11C highlights DNA fragments of about 50 bp.

FIGS. 12A and 12B show the qPCR result of a single-stranded DNA fragment isolated by the magnetic nanoparticles described herein and by a commercially available magnetic bead as compared to the original single-stranded DNA fragment sample, with FIG. 12A showing the amplification plot; and FIG. 12B showing the DNA yield as represented by C_{T} value.

FIG. 13 shows the purification result of an RNA ladder sample (100-1000 nt) spiked in serum isolated by contact with and elution from the magnetic nanoparticles described herein (solid line) as compared to the original RNA ladder (dotted line). The RNA samples were analyzed by an Agilent 2100 bioanalyzer.

FIG. 14 shows the purification result of a small RNA ladder sample (17-150 nt) spiked in plasma isolated by contact with and elution from the magnetic nanoparticles described herein (solid line) as compared to the original small RNA ladder (dotted line). The RNA samples were analyzed by an Agilent 2100 bioanalyzer.

FIGS. 15A, 15B, and 15C show the qPCR results of a synthetic RNA mimic cel-miR-39 isolated by the magnetic nanoparticles described herein (solid line) and by a commercially available column-based RNA isolation product (dotted line), with FIG. 15A showing the amplification plot and ΔC_{T} value of cfRNA isolated from blood collected in K₃EDTA BCT; FIG. 15B showing the amplification plot and ΔC_{T} value of cfRNA isolated from blood collected in cfRNA BCT vendor 1; and FIG. 15C showing the amplification plot and ΔC_{T} value of cfRNA isolated from blood collected in cfRNA BCT vendor 2.

FIGS. 16A, 16B, 16C, and 16D show the qPCR results of endogenous cfRNA isolated by the magnetic nanoparticles described herein (solid line) and by a commercially available column-based RNA isolation product (dotted line) from blood collect in K₃EDTA BCT, with FIG. 16A showing the amplification plot and ΔC_{T} value of beta-globin; FIG. 16B showing the amplification plot and ΔC_{T} value of miR-21; FIG. 16C showing the amplification plot and ΔC_{T} value of U6; and FIG. 16D showing the amplification plot and ΔC_{T} value of miR-15a.

FIGS. 17A, 17B, 17C, and 17D show the qPCR results of endogenous cfRNA isolated by the magnetic nanoparticles described herein (solid line) and by a commercially available column-based RNA isolation product (dotted line) from blood collect in cfRNA BCT vendor 1, with FIG. 17A showing the amplification plot and ΔC_{T} value of beta-globin; FIG. 17B showing the amplification plot and ΔC_{T} value of miR-21; FIG. 17C showing the amplification plot and ΔC_{T} value of U6; and FIG. 17D showing the amplification plot and ΔC_{T} value of miR-15a.

FIGS. 18A, 18B, 18C, and 18D show the qPCR results of endogenous cfRNA isolated by the magnetic nanoparticles described herein (solid line) and by a commercially available column-based RNA isolation product (dotted line) from blood collect in cfRNA BCT vendor 2, with FIG. 18A showing the amplification plot and ΔC_{T} value of beta-globin;

FIG. 18B showing the amplification plot and ΔC_{T} value of miR-21; FIG. 18C showing the amplification plot and ΔC_{T} value of U6; and FIG. 18D showing the amplification plot and ΔC_{T} value of miR-15a.

### DETAILED DESCRIPTION

### Definitions

Terms used herein have the meanings ascribed by persons of ordinary skill in the art, unless otherwise defined as set forth below.

As used herein, the term “single core” means that there is only one magnetic core in each magnetic particle. Single core magnetic particles do not include magnetic beads with interspersed small magnetic pieces or magnetic beads with more than one magnetic core.

The term “maximum diameter” refers to the maximum distance between two antipodal points on the surface of a particle; as used herein “the same maximum diameter” means the difference between two maximum diameters is no more than 20%.

As used herein, the term “mesoporous” refers to a structure containing pores with diameters between 1 and 50 nm. Mesoporous structure can be generated from silica or alumina materials with surfactant self-assembly. The mesopores can be similarly-sized and differently-sized.

As used herein, the term “functional group” refers to a chemical group bound to the surface of the magnetic nanoparticle. The functional group can be linked to the outer shell of the magnetic nanoparticle covalently or non-covalently. The functional group can bind to a specific target molecule, or to a molecular probe that can bind specifically to a target molecule. Suitable functional groups include but are not limited to carboxyl, hydroxyl, epoxy, carbonyl, aldehyde, amine, maleimide, N-hydroxysuccinimide, carbodiimide, anhydride, hydrazide, biotin, polyethylene glycol, azide, nitrile, sulfhydryl, thiocyanate, phosphate, borono, thioester, cysteine, disulfide, alkyl and acyl halide, glutathione, maltose, isocyanate, sulfonyl chloride, tosylate ester, carbonate, arylating agent, imidoester, fluorophenyl ester, and Schiff base.

As used herein, the term “molecular probe” refers to a molecule or a group of molecules that are used to study the properties of other molecules. The molecular probe comprises a functional moiety, which can bind to a target molecule. The molecular probe can further comprise a moiety capable of binding to the magnetic nanoparticle. A molecular probe can be a nucleic acid probe or a protein probe. A nucleic acid probe comprises a nucleic acid functional moiety, which comprises a complementary sequence to a portion of a target polynucleotide sequence.

As used herein, the term “hybridize specifically” refer to a single stranded polynecleotide, such as single stranded DNA or RNA, annealing to a complementary DNA or RNA. The term “hybridize specifically” includes the interaction between a specifically designed probe and its desired target, such as the binding of polydT specifically to all mRNAs with a polyA tail.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term “antibody” is meant to include whole antibodies, polyclonal, monoclonal and recombinant antibodies, fragments thereof, and further includes single-chain antibodies, human antibodies, humanized antibodies, murine antibodies, chimeric, mouse-human, mouse-primate, primate-human monoclonal antibodies, anti-idiotype antibodies, antibody fragments, such as, scFv, (scFv)₂, Fab, Fab′, F(ab′)₂, Fv, dAb, Nanobody, Fd fragments, diabodies, and antibody-related polypeptides. Antibody includes bispecific antibodies and multispecific antibodies so long as they exhibit the desired biological activity or function.

As used herein, the terms “enrich,” “enriched,” and “enriching” or “isolate,” “isolated,” and “isolating” or “purify,” “purified,” and “purifying” or “extract,” “extracted,” and “extracting” are used interchangeably and refer to the state of a preparation (e.g., a plurality of known or unknown amount and/or concentration) of desired target molecules, that have undergone one or more processes of purification. In some embodiments, enriching, isolating, or purifying as used herein is the process of removing, partially removing contaminants from a staring sample. In some embodiments, an isolated target molecule has no detectable undesired activity or, alternatively, the level or amount of the undesired activity is at or below an acceptable level or amount. In other embodiments, an isolated composition has an amount and/or concentration of target molecule at or above an acceptable amount and/or concentration. In other embodiments, the isolated target molecule composition is enriched as compared to the starting material from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some embodiments, the target molecule compositions are substantially free of residual biological products. In some embodiments, the isolated target molecule preparations are 100% free, 99% free, 98% free, 97% free, 96% free, or 95% free of any contaminating biological matter. Residual biological products can include abiotic materials (including chemicals) or unwanted nucleic acids, proteins, lipids, or metabolites.

The term “sample” is defined as a representative part or a small amount from a larger whole that can provide information about the whole that it is taken from. A “sample” includes an aliquot of a sample.

As used herein, the terms “circulating free DNA (cfDNA),” “cell-free DNA,” and “circulating cell-free DNA” are used interchangeably and refer to small DNA fragments found circulating in plasma or serum, as well as other bodily fluids. During pregnancy, illness, and exacerbation of tissue (intensive exercise or injury) the levels of cfDNA generally increase. Elevated levels of cfDNA are observed in cancer, especially in advanced disease. The detection of increased levels of cfDNA during pregnancy and diseases has potential application as a non-invasive method for diagnosis and monitoring of disease.

The term “circulating tumor DNA (ctDNA)” refers to cfDNA that is shed from tumor cells into the circulatory system. ctDNA can originate from the tumor or from circulating tumor cells (CTCs).

The term “circulating tumor cells (CTCs)” refers to cells that are shed into the circulatory system, such as vasculature or lymphatics from a primary tumor and are carried around the body in the circulation. CTCs can serve as seeds for subsequent growth of additional tumors in distant organs, known as metastasis. CTCs can be used as “liquid biopsy” which reveals metastasis and provides information about the patient's disease status.

The term “chaotropic agent” refers to a substance which disrupts the structure of, and denatures, macromolecules such as proteins and nucleic acids (e.g. DNA and RNA). Chaotropic agents interfere with intramolecular interactions mediated by non-covalent forces such as hydrogen bonds, van der Waals forces, and hydrophobic effects. Chaotropic agents include but are not limited to guanidine salt, sodium iodide, potassium iodide, sodium thiocyanate, sodium isothiocyanate, urea, and combinations thereof. In some embodiments, the chaotropic agent is a sodium salt or guanidinium salt, preferably sodium iodide, sodium perchlorate, guanidinium hydrochloride, guanidinium thiocyanate, guanidinium isothiocyanate, or a mixture of two or more salts thereof. In some embodiments, the chaotropic agent is a guanidinium salt, preferably guanidinium hydrochloride, guanidinium thiocyanate, or guanidinium isothiocyanate, or a mixture of two or more salts thereof. In some embodiments, the chaotropic agent is not needed for the interaction between the magnetic nanoparticle and its target molecule, such as the interaction between an antibody conjugated magnetic nanoparticle and its target protein, or the interaction between a poly nucleotide conjugated magnetic nanoparticle and its target DNA or RNA.

The term “organic solvent” refers carbon based solvents that are capable of dissolving other substances. Examples of organic solvent include but are not limited to ethylene glycol, polyethylene glycol, ethanol, methanol, propanol, isopropanol, propylene glycol, and poly propylene glycol.

As used herein, the term “capping agent” refers to an agent used in synthesis of nanoparticle that inhibits nanoparticle overgrowth and aggregation. Capping agent also influences the structure characteristics of the resulted nanoparticles. Suitable capping agents include but are not limited to polyethylene glycol, polypropylene glycol, acetate ions, citrate ions, formate ions, propionic ions, and succinate ions.

The term “surfactant” refers to a compound that lowers the surface tension between two lipids or between a lipid and a solid. Surfactants can be amphiphilic organic compounds with both hydrophobic groups and hydrophilic groups. Surfactants can be ionic or non-ionic. Ionic surfactants include but are not limited to alkylbenzene sulfonate, fatty acid soap, lauryl sulfate, di-alkyl sulfosuccinate, lignosulfonate, fatty amine salt and quaternary ammonium. Non-ionic surfactants include but are not limited to polyoxyethylene fatty alcohol ether, polyoxyethylene alkylphenyl ether and polyoxyethylene-polyoxypropylene block copolymers.

1.1. The Magnetic Nanoparticle

In a first aspect, disclosed herein is a magnetic nanoparticle. In typical embodiments, the magnetic nanoparticle comprises a single magnetic core and an outer shell. The magnetic core makes the magnetic nanoparticle responsive to external magnetic force. In some of these embodiments, the outer shell covers the magnetic core. In certain embodiments, the outer shell protects the magnetic core. In certain embodiments, the outer shell prevents leaking of magnetic material from the magnetic core. In certain embodiments, the outer shell provides surface chemistry for binding of target molecules.

In some embodiments, the magnetic nanoparticle has a maximum diameter of less than 1 μm, such as less than 900 nm, less than 800 nm, less than 700 nm, less than 600 nm, less than 500 nm, less than 400 nm, less than 300 nm, or less than 200 nm. In various embodiments, the magnetic nanoparticle has a maximum diameter of 100 nm to 1000 nm, such as 100 nm to 900 nm, 100 nm to 800 nm, 100 nm to 700 nm, 100 nm to 600 nm, 100 nm to 500 nm, 100 nm to 400 nm, 100 nm to 300 nm, 100 nm to 200 nm, 200 nm to 1000 nm, 200 nm to 900 nm, 200 nm to 800 nm, 200 nm to 700 nm, 200 nm to 600 nm, 200 nm to 500 nm, 200 nm to 400 nm, 200 nm to 300 nm, 300 nm to 1000 nm, 300 nm to 900 nm, 300 nm to 800 nm, 300 nm to 700 nm, 300 nm to 600 nm, 300 nm to 500 nm, 300 nm to 400 nm, 400 nm to 1000 nm, 400 nm to 900 nm, 400 nm to 800 nm, 400 nm to 700 nm, 400 nm to 600 nm, 400 nm to 500 nm, 500 nm to 1000 nm, 500 nm to 900 nm, 500 nm to 800 nm, 500 nm to 700 nm, 500 nm to 600 nm, 600 nm to 1000 nm, 600 nm to 900 nm, 600 nm to 800 nm, 600 nm to 700 nm, 700 nm to 1000 nm, 700 nm to 900 nm, 700 nm to 800 nm, 800 nm to 1000 nm, 800 nm to 900 nm, or 900 nm to 1000 nm. In certain embodiments, the magnetic core has a maximum diameter of about 900 nm. In certain embodiments, the magnetic core has a maximum diameter of about 800 nm. In certain embodiments, the magnetic core has a maximum diameter of about 700 nm. In certain embodiments, the magnetic core has a maximum diameter of about 600 nm. In certain embodiments, the magnetic core has a maximum diameter of about 500 nm. In certain embodiments, the magnetic core has a maximum diameter of about 400 nm. In certain embodiments, the magnetic core has a maximum diameter of about 300 nm. In certain embodiments, the magnetic core has a maximum diameter of about 200 nm.

Unless otherwise specified, the maximum diameter of the magnetic nanoparticle is measured by dynamic light scattering.

In various embodiments, the size and structure of the magnetic nanoparticle allow it to remain dispersed in an aqueous medium for a time sufficient to permit the binding between the magnetic nanoparticle and the target molecule.

1.1.1 The Core

In various embodiments, the magnetic core comprises a cluster of magnetic crystals. In currently preferred embodiments, the cluster of magnetic crystals forms a single core. In some embodiments, the magnetic core is composed of metal oxide. In some of these embodiments, the metal oxide is an iron oxide. In specific embodiments, the iron oxide is Fe₃O₄. In some embodiments, the magnetic core has a composition of XFe₂O₄. In various embodiments, X can be Mn, Ca, Co, Zn, Cu, Mg, Ba, Ni, or Cr.

In some embodiments, the magnetic core has a maximum diameter of less than 1 μm, such as less than 800 nm, less than 700 nm, less than 600 nm, less than 500 nm, less than 400 nm, less than 300 nm, or less than 200 nm. In various embodiments, the magnetic core has a maximum diameter of 100 nm to 800 nm, such as 100 nm to 700 nm, 100 nm to 600 nm, 100 nm to 500 nm, 100 nm to 400 nm, 100 nm to 300 nm, 100 nm to 200 nm, 200 nm to 800 nm, 200 nm to 700 nm, 200 nm to 600 nm, 200 nm to 500 nm, 200 nm to 400 nm, 200 nm to 300 nm, 300 nm to 800 nm, 300 nm to 700 nm, 300 nm to 600 nm, 300 nm to 500 nm, 300 nm to 400 nm, 400 nm to 800 nm, 400 nm to 700 nm, 400 nm to 600 nm, 400 nm to 500 nm, 500 nm to 800 nm, 500 nm to 700 nm, 500 nm to 600 nm, 600 nm to 800 nm, 600 nm to 700 nm, or 700 nm to 800 nm. In certain embodiments, the magnetic core has a maximum diameter of about 700 nm. In certain embodiments, the magnetic core has a maximum diameter of about 600 nm. In certain embodiments, the magnetic core has a maximum diameter of about 500 nm. In certain embodiments, the magnetic core has a maximum diameter of about 450 nm. In certain embodiments, the magnetic core has a maximum diameter of about 400 nm. In certain embodiments, the magnetic core has a maximum diameter of about 350 nm. In certain embodiments, the magnetic core has a maximum diameter of about 300 nm. In certain embodiments, the magnetic core has a maximum diameter of about 250 nm. In certain embodiments, the magnetic core has a maximum diameter of about 200 nm.

Unless otherwise specified, the maximum diameter of the magnetic core is measured by dynamic light scattering.

1.1.2 The Shell

In various embodiments, the outer shell comprises silicon dioxide, titanium dioxide, or polymer. In some embodiments, the outer shell comprises silicon dioxide. In certain embodiments, the silicon dioxide is amorphous. In certain other embodiments, the silicon dioxide is in crystallized form. In some embodiments, the outer shell comprises titanium dioxide. In certain embodiments, the titanium dioxide is amorphous. In certain other embodiments, the titanium dioxide is in crystallized form.

In some embodiments, the outer shell comprises polymer, such as polyethylene glycol, polyacrylic acid, polyacrylamide, polyvinyl alcohol, poly-methyl methacrylate, polystyrene, poly-4-vinylphenol, polyester, polyimide, polyethylene, polypropylene, polyethylene vinyl acetate, polyacrylates, polysaccharide, etc. In some embodiments, the polymer has different molecular weight. In various embodiments, the polymer has an average molecular weight of 100 to 500000 Dalton, such as 200 to 100000 Dalton, 300 to 50000 Dalton, 400 to 20000 Dalton, 500 to 10000 Dalton, or 600 to 5000 Dalton. In some embodiments, the polymer comprises one monomer unit. In some of these embodiments, the monomer is ethylene glycol, acrylic acid, acrylamide, or styrene. In some embodiments, the polymer is a copolymer comprising two or more different monomer units. In some of these embodiments, the two or more different monomer units are selected from ethylene glycol, acrylic acid, acrylamide, and styrene. In some embodiments, the polymer has a linear structure. In some embodiments, the polymer has a branched structure. In some embodiments, the polymer is cross-linked.

In some embodiments, the outer shell has a thickness of less than 300 nm, such as less than 250 nm, less than 200 nm, less than 150 nm, less than 100 nm, less than 80 nm, less than 50 nm, less than 20 nm, less than 10 nm, less than 5 nm, less than 2 nm, or less than 1 nm. In various embodiments, the outer shell has a thickness of 1 nm to 300 nm, such as 1 nm to 250 nm, 1 nm to 200 nm, 1 nm to 150 nm, 1 nm to 100 nm, 1 nm to 50 nm, 1 nm to 10 nm, 10 nm to 300 nm, 10 nm to 250 nm, 10 nm to 200 nm, 10 nm to 150 nm, 10 nm to 100 nm, 10 nm to 50 nm, 50 nm to 300 nm, 50 nm to 250 nm, 50 nm to 200 nm, 50 nm to 150 nm, 50 nm to 100 nm, 100 nm to 300 nm, 100 nm to 250 nm, 100 nm to 200 nm, 100 nm to 150 nm, 150 nm to 300 nm, 150 nm to 250 nm, 150 nm to 200 nm, 200 nm to 300 nm, 200 nm to 250 nm, or 250 nm to 300 nm. In certain embodiments, the outer shell has a thickness of about 250 nm. In certain embodiments, the outer shell has a thickness of about 200 nm. In certain embodiments, the outer shell has a thickness of about 150 nm. In certain embodiments, the outer shell has a thickness of about 100 nm. In certain embodiments, the outer shell has a thickness of about 80 nm. In certain embodiments, the outer shell has a thickness of about 60 nm. In certain embodiments, the outer shell has a thickness of about 40 nm. In certain embodiments, the outer shell has a thickness of about 20 nm. In certain embodiments, the outer shell has a thickness of about 10 nm. In certain embodiments, the outer shell has a thickness of about 5 nm. In certain embodiments, the outer shell has a thickness of about 2 nm. In certain embodiments, the outer shell has a thickness of about 1 nm.

Unless otherwise specified, the thickness of the outer shell is measured by subtracting the diameter of the magnetic core, as measured by dynamic light scattering from the diameter of the magnetic nanoparticle, as measured by dynamic light scattering.

In some embodiments, the shell is a single layer. In other embodiments, the shell comprises a plurality of layers. In certain embodiments, the shell comprises a layer of silicon dioxide and a layer of titanium dioxide. In certain embodiments, the shell comprises a layer of silicon dioxide and a layer of polymer. In certain embodiments, the shell comprises a layer of titanium dioxide and a layer of polymer.

In some embodiments, the shell comprises at least one nonporous layer. In certain embodiments, the shell comprises a nonporous silicon dioxide layer. In certain embodiments, the shell comprises a nonporous titanium dioxide layer. In certain embodiments, the shell comprises a nonporous polymer layer. In certain embodiments, the shell comprises a layer of nonporous silicon dioxide and a layer of nonporous titanium dioxide. In certain embodiments, the shell comprises a layer of nonporous silicon dioxide and a layer of nonporous polymer. In certain embodiments, the shell comprises a layer of nonporous titanium dioxide and a layer of nonporous polymer.

1.1.2.1 The Mesoporous Structure

In certain embodiments, the outer shell comprises at least one layer with mesoporous structure.

In certain embodiments, the outer shell comprises a layer of mesoporous silicon dioxide. In certain embodiments, the outer shell comprises a layer of mesoporous titanium dioxide. In certain embodiments, the outer shell comprises a layer of mesoporous polymer.

In some embodiments, the outer shell comprises a layer of nonporous material and a layer of mesoporous material. In some of these embodiments, the layer of mesoporous material covers the layer of nonporous material. In some embodiments, the outer shell comprises a layer of nonporous silicon dioxide and a layer of mesoporous silicon dioxide. In some of these embodiments, the layer of mesoporous silicon dioxide covers the layer of nonporous silicon dioxide. In some embodiments, the outer shell comprises a layer of nonporous titanium dioxide and a layer of mesoporous titanium dioxide. In some of these embodiments, the layer of mesoporous titanium dioxide covers the layer of nonporous titanium dioxide. In some embodiments, the outer shell comprises a layer of nonporous polymer and a layer of mesoporous polymer. In some of these embodiments, the layer of mesoporous polymer covers the layer of nonporous polymer. In some embodiments, the outer shell comprises a layer of nonporous silicon dioxide and a layer of mesoporous titanium dioxide. In some of these embodiments, the layer of mesoporous titanium dioxide covers the layer of nonporous silicon dioxide. In some embodiments, the outer shell comprises a layer of nonporous titanium dioxide and a layer of mesoporous silicon dioxide. In some of these embodiments, the layer of mesoporous silicon dioxide covers the layer of nonporous titanium dioxide. In some embodiments, the outer shell comprises a layer of nonporous polymer and a layer of mesoporous silicon dioxide. In some of these embodiments, the layer of mesoporous silicon dioxide covers the layer of nonporous polymer. In some embodiments, the outer shell comprises a layer of nonporous silicon dioxide and a layer of mesoporous polymer. In some of these embodiments, the layer of mesoporous polymer covers the layer of nonporous silicon dioxide. In some embodiments, the outer shell comprises a layer of nonporous polymer and a layer of mesoporous titanium dioxide. In some of these embodiments, the layer of mesoporous titanium dioxide covers the layer of nonporous polymer. In some embodiments, the outer shell comprises a layer of nonporous titanium dioxide and a layer of mesoporous polymer. In some of these embodiments, the layer of mesoporous polymer covers the layer of nonporous titanium dioxide.

In some embodiments, the mesoporous structure has an average surface pore diameter of less than 50 nm, such as less than 30 nm, less than 20 nm, less than 10 nm, less than 5 nm, or less than 2 nm. In various embodiments, the mesoporous structure has an average surface pore diameter of 1 nm to 50 nm, such as 1 nm to 30 nm, 1 nm to 20 nm, 1 nm to 10 nm, 1 nm to 5 nm, 1 nm to 2 nm, 2 nm to 50 nm, 2 nm to 30 nm, 2 nm to 20 nm, 2 nm to 10 nm, 2 nm to 5 nm, 5 nm to 50 nm, 5 nm to 30 nm, 5 nm to 20 nm, 5 nm to 10 nm, 10 nm to 50 nm, 10 nm to 30 nm, 10 nm to 20 nm, 20 nm to 50 nm, 20 nm to 30 nm, or 30 nm to 50 nm. In certain embodiments, the mesoporous structure has an average surface pore diameter of about 30 nm. In certain embodiments, the mesoporous structure has an average surface pore diameter of about 20 nm. In certain embodiments, the mesoporous structure has an average surface pore diameter of about 10 nm. In certain embodiments, the mesoporous structure has an average surface pore diameter of about 5 nm. In certain embodiments, the mesoporous structure has an average surface pore diameter of about 2 nm.

Unless otherwise specified, the surface pore diameter is measured by transmission electron microscopy.

1.1.3 The Functional Group

In various embodiments, the magnetic nanoparticle comprises at least one species of functional group.

In typical embodiments, the functional group is attached to the outer shell. In some embodiments, the functional group is covalently attached to the outer shell. In some embodiments, the functional group is attached to the outer shell non-covalently. In some embodiments, the functional group is capable of binding directly to a target molecule, such as a nucleic acid, a protein, a peptide, a carbohydrate, a lipid, or an organic molecule. In certain embodiments, the functional group is capable of binding to a molecular probe, such as a nucleic acid probe or a protein probe. In some of these latter embodiments, the molecular probe is capable, in turn, of binding to a target molecule, such as a nucleic acid, a protein, a peptide, a carbohydrate, or a lipid.

In various embodiments, the functional group is carboxyl, hydroxyl, epoxy, carbonyl, aldehyde, amine, maleimide, N-hydroxysuccinimide, carbodiimide, anhydride, hydrazide, polyethylene glycol, azide, nitrile, sulfhydryl, thiocyanate, phosphate, borono, thioester, cysteine, disulfide, alkyl and acyl halide, glutathione, maltose, isocyanate, sulfonyl chloride, tosylate ester, carbonate, arylating agent, imidoester, fluorophenyl ester, or Schiff base. In some embodiments, the functional group can be carboxyl, hydroxyl, epoxy, carbonyl, aldehyde, amine, maleimide, N-hydroxysuccinimide, carbodiimide, anhydride, hydrazide, or biotin.

In some embodiments, the magnetic particle comprises a plurality of functional group species. In typical embodiments, each of the plurality of functional groups is attached to the outer shell.

In various embodiments, the functional group lead to the creation of surface charge of the magnetic nanoparticle. In some embodiments, the surface charge of the magnetic nanoparticle is positive. In some embodiments, the surface charge of the magnetic nanoparticle is negative. In some embodiments, the surface charge of the magnetic nanoparticle can be tuned by changing the pH of the solution.

1.1.4 Additional Functional Moiety

In various embodiments, the magnetic nanoparticle further comprises at least one species of additional functional moiety.

In certain embodiments, the functional moiety is a polynucleotide, a polysaccharide, a polypeptide, a protein, an aptamer, or an ion. In some embodiments, the polynucleotide, the polysaccharide, the polypeptide, the protein, the aptamer, or the ion is covalently bound to the outer shell. In some embodiments, the polynucleotide, the polysaccharide, the polypeptide, the protein, the aptamer, or the ion is non-covalently bound to the outer shell. In some embodiments, the polynucleotide, the polysaccharide, the polypeptide, the protein, the aptamer, or the ion is covalently bound to the functional group. In some embodiments, the polynucleotide, the polysaccharide, the polypeptide, the protein, the aptamer, or the ion is non-covalently bound to the functional group.

In some embodiments, the additional functional moiety is polynucleotide. In various embodiments, the polynucleotide has a length of 5 to 200 bases, such as 5 to 150 bases, 5 to 100 bases, 5 to 80 bases, 5 to 50 bases, 5 to 30 bases, 5 to 20 bases, 5 to 10 bases, 10 to 200 bases, 10 to 150 bases, 10 to 100 bases, 10 to 80 bases, 10 to 50 bases, 10 to 30 bases, 10 to 20 bases, 20 to 200 bases, 20 to 150 bases, 20 to 100 bases, 20 to 80 bases, 20 to 50 bases, 20 to 30 bases, 30 to 200 bases, 30 to 150 bases, 30 to 100 bases, 30 to 80 bases, 30 to 50 bases, 50 to 200 bases, 50 to 150 bases, 50 to 100 bases, 50 to 80 bases, 80 to 200 bases, 80 to 150 bases, 80 to 100 bases, 100 to 200 bases, 100 to 150 bases, or 150 to 200 bases. In certain embodiments, the polynucleotide can bind to a protein target, a DNA target, or an RNA target. In some embodiments, the polynucleotide has a specific, predetermined, sequence. In typical embodiments, the predetermined sequence is at least partly complementary to the sequence of a nucleic acid target and the polynucleotide moiety hybridizes specifically with a target DNA or RNA molecule.

In some embodiments, the magnetic nanoparticle comprises a plurality of polynucleotide species, each of the plurality having a different predetermined sequence.

In various embodiments, the target DNA molecule is a double stranded DNA (dsDNA), single stranded DNA (ssDNA), or a combination thereof. In specific embodiments, the target DNA molecule is circulating free DNA (cfDNA). In various embodiments, the target RNA molecule is an mRNA, an rRNA, a tRNA, a lncRNA, a miRNA, an siRNA, an shRNA, or a combination thereof. In specific embodiments, the polynucleotide is polydT. In some of these embodiments, the polydT hybridizes specifically with the polyA tail of an mRNA.

In various embodiments, the polynucleotide further comprises biotin.

In some embodiments, the additional functional moiety is a molecular probe.

In some embodiments, the additional functional moiety is a polypeptide or a protein. In certain embodiments, the protein is a biotin-binding protein, such as streptavidin, avidin or NeutrAvidin. In certain embodiments, the polypeptide or the protein is an antibody or an antigen-binding fragment, including but not limited to scFv, (scFv)₂, Fab, Fab′, F(ab′)₂, Fv, dAb, Nanobody, Fd fragments, diabodies, and antibody-related polypeptides. In some of these embodiments, the antibody or an antigen-binding fragment further comprises biotin. In certain embodiments, the protein is an antibody-binding protein, such as Protein A, Protein G, Protein A/G or Protein L. In some of these embodiments, the antibody-binding protein further comprises biotin.

In some embodiments, the functional moiety is a chemical bound. In certain embodiments, the chemical bound is a reversible chemical bound.

1.2 Compositions

Also provided herein are compositions comprising a plurality of magnetic nanoparticles.

In certain embodiments, the plurality of magnetic nanoparticles are in solid state. In certain embodiments, the plurality of magnetic nanoparticles are dispersed in a liquid. In some embodiments, the composition further comprises an aqueous solution. In various embodiments, the aqueous solution is water, ethanol, isopropanol, TE buffer, PBS, PBS-Tween20®, TBS, or TBS-Tween20®.

In various embodiments, at least 40% of the magnetic nanoparticles within the composition have the same maximum diameter. In some embodiments, at least 50% of the magnetic nanoparticles have the same maximum diameter. In some embodiments, at least 60% of the magnetic nanoparticles have the same maximum diameter. In some embodiments, at least 70% of the magnetic nanoparticles have the same maximum diameter. In some embodiments, at least 80% of the magnetic nanoparticles have the same maximum diameter. In some embodiments, at least 90% of the magnetic nanoparticles have the same maximum diameter. In some embodiments, at least 95% of the magnetic nanoparticles have the same maximum diameter.

Unless otherwise specified, the size distribution of the magnetic nanoparticles is measured by dynamic light scattering particle size analyzer.

1.3 The Kit

Also provided herein are kits for isolating biological targets. The kits comprise compositions of magnetic nanoparticles as described above. In some embodiments, the kit further comprises a buffer. In some embodiments, the kit further comprises a combination of buffers. Suitable buffers include but are not limited to lysis buffer, suspension buffer, precipitation buffer, binding buffer, labeling buffer, washing buffer, and elution buffer.

In some embodiments, the kit further comprises a chaotropic agent. In certain embodiments, the chaotropic agent disrupts the hydrogen bond between the target molecule and the surrounding solvent molecules. In certain embodiments, the chaotropic agent increases the affinity of the target molecule to the magnetic nanoparticle. In various embodiments, the chaotropic agent is guanidine salt, sodium iodide, potassium iodide, sodium thiocyanate, sodium isothiocyanate, urea, or combinations thereof. In some embodiments, the chaotropic agent is a sodium salt or guanidinium salt, preferably sodium iodide, sodium perchlorate, guanidinium hydrochloride, guanidinium thiocyanate, guanidinium isothiocyanate, or a mixture of two or more salts thereof. In some embodiments, the chaotropic agent is a guanidinium salt, preferably guanidinium hydrochloride, guanidinium thiocyanate, or guanidinium isothiocyanate, or a mixture of two or more salts thereof.

1.4 Methods of Enriching Biological Target

Also disclosed herein are methods of enriching one or more molecular targets from a biological medium. The methods comprise: a) providing a sample of biological medium containing or suspected of containing one or more biological targets; b) adding to the sample a composition comprising dispersed magnetic nanoparticles capable of binding the biological target under conditions that permit a complex to form between the magnetic nanoparticle and the biological target; c) separating the complex from the biological medium by application of an external magnetic field; and d) recovering the biological target from the magnetic nanoparticles.

In some embodiments, the magnetic nanoparticle does not comprise an additional functional moiety. In some of these embodiments, the method comprises: providing a sample of biological medium containing one or more biological target; adding to the sample the molecular probes capable of binding the biological target under conditions that permit a complex to form between the molecular probe and the biological target; adding the composition comprising dispersed magnetic nanoparticles capable of binding the molecular probe conditions that permit a complex to form between the magnetic nanoparticle and molecular probe; separating the magnetic nanoparticle-molecular probe-biological target complex from the biological medium by application of an external magnetic field; and recovering the biological target. In some of these embodiments, the molecular probes are added to the sample prior to addition of the magnetic nanoparticle composition. In some embodiments, the molecular probes are added to the sample concurrently with addition of the magnetic nanoparticle composition. In various embodiments, the molecular probe comprises a moiety with high affinity for a molecule on the magnetic nanoparticle.

In some embodiments, the magnetic particle comprises an additional functional moiety. In particular embodiments, the additional functional moiety is a molecular probe. In specific embodiments, the method comprises: providing a sample of biological medium containing one or more biological targets; adding to the sample a composition comprising dispersed magnetic nanoparticles comprising molecular probes capable of binding the biological target under conditions that permit a complex to form between the molecular probe and the biological target; separating the magnetic nanoparticle-molecular probe-biological target complex from the biological medium by application of an external magnetic field; and recovering the biological target.

In various embodiments, the biological targets include but are not limited to nucleic acids, peptides, proteins, carbohydrates, lipids, organic or inorganic biomolecules, cells, cellular organelles, and viruses. In certain embodiments, the biological target can be a nucleic acid, a peptide, a protein, a carbohydrate, a lipid, a cell, or a cellular organelle, such as an exosome.

In some embodiments, the method further comprises pretreating the sample of biological medium to effect the release of the biological target. In certain embodiments, the pretreatment comprises disrupting the biological medium to release nucleic acids, peptides, proteins, carbohydrates, lipids, and/or other organic or inorganic biological molecules. In certain embodiments, the pretreatment comprises disrupting the biological medium to release one or more cells or cellular organelles. In certain embodiments, the method further comprises centrifuging the biological medium to remove one or more cells or cellular organelles. In certain embodiments, the pretreatment comprises removing contaminants to minimize their effect on enriching the target. For example, protein contaminants can be removed by protein precipitation or protein digestion.

In various embodiments, the biological medium is a body fluid. In some embodiments, the body fluid is used as a liquid biopsy for diagnosing and monitoring diseases, such as cancer. In some embodiments, the body fluid is blood, serum, plasma, saliva, cerebrospinal fluid, urine, semen, or ascites. In specific embodiments, the body fluid is blood, serum, or plasma. In certain embodiments, the body fluid is collected from a healthy individual. In certain embodiments, the body fluid is collected from an individual with, or suspected of having, a disease. In some embodiments, the body fluid is collected from a patient with, or suspected of having, cancer. In some embodiments, the body fluid is collected from a patient with, or suspected of having, an infectious disease. In some embodiments, the body fluid is collected from a pregnant woman.

1.4.1 Nucleic Acid Target

In various embodiments, the biological target is a nucleic acid, such as DNA or RNA. In certain embodiments, the DNA or RNA targets present in the sample are first modified by biotin. In some of these embodiments, the magnetic nanoparticle comprises a biotin-binding protein, such as streptavidin, avidin, or NeutrAvidin. In certain embodiments, the target DNA or RNA comprises a sequence that can hybridize specifically with a nucleic acid functional moiety on the surface of the magnetic nanoparticle.

In some embodiments, the biological target is a DNA molecule. In some of these embodiments, the biological target is a double stranded DNA (dsDNA). In some of these embodiments, the biological target is a single stranded DNA (ssDNA). In yet some of these embodiments, the biological target is a combination of dsDNA and ssDNA.

In certain embodiments, the target DNA molecular has a length of less than 500 bp, such as less than 400 bp, less than 300 bp, less than 200 bp, less than 100 bp, or less than 50 bp.

In some embodiments, the biological target is a cellular DNA. In some embodiments, the biological target is a circulating free DNA (cfDNA). In some of these embodiments, the biological target is a cfDNA from a patient with, or suspected of having, cancer. In some embodiments, the biological target is a circulating tumor DNA (ctDNA). In certain embodiments, the cfDNA or the ctDNA is isolated from the blood, serum, or plasma of a patient. In some embodiments, the biological target is a virus DNA. In some of these embodiments, the biological target is a virus DNA from a patient with, or suspected of having, an infectious disease. In certain embodiments, the virus DNA is isolated from the blood, serum, or plasma of a patient. In some embodiments, the biological target is a fetal DNA. In some of these embodiments, the biological target is a fetal DNA from a pregnant woman. In certain embodiments, the fetal DNA is isolated from the blood, serum, or plasma of a pregnant women.

In some embodiments, the biological target is an RNA molecule. In various embodiments, the biological target is an mRNA, an rRNA, a tRNA, a lncRNA, a miRNA, an siRNA, an shRNA, or a combination thereof. In specific embodiments, the biological target is an mRNA. In some of these embodiments, the polyA tail of the mRNA hybridizes specifically with the polydT on the surface of the magnetic nanoparticle.

In certain embodiments, the target RNA molecular has a length of less than 500 nt, such as less than 400 nt, less than 300 nt, less than 200 nt, less than 100 nt, or less than 50 nt.

In some embodiments, the biological target is a cellular RNA. In some embodiments, the biological target is a circulating free RNA (cfRNA). In some of these embodiments, the biological target is a cfRNA from a patient with, or suspected of having, cancer. In certain embodiments, the cfRNA is isolated from the blood, serum, or plasma of a patient. In some embodiments, the biological target is a virus RNA. In some of these embodiments, the biological target is a virus RNA from a patient with, or suspected of having, an infectious disease. In certain embodiments, the virus RNA is isolated from the blood, serum, or plasma of a patient. In some embodiments, the biological target is a fetal RNA. In some of these embodiments, the biological target is a fetal RNA from a pregnant woman. In certain embodiments, the fetal RNA is isolated from the blood, serum, or plasma of a pregnant women.

1.4.2 Protein Target

In various embodiments, the biological target is a peptide or a protein. In some of these embodiments, the magnetic nanoparticle comprises a biomolecule which binds to the target peptide or protein. In certain embodiments, the peptide or protein target in the sample is first modified by biotin. In some of these embodiments, the magnetic nanoparticle comprises a biotin-binding protein, such as streptavidin, avidin or NeutrAvidin.

In certain embodiments, the biological target is an antibody. In some of these embodiments, the magnetic nanoparticle comprises an antibody-binding protein, such as Protein A, Protein G, Protein A/G or Protein L. In certain embodiments, the antibody-binding protein further comprises biotin. In certain embodiments, the biological target is a peptide or protein capable of binding to an antibody, or an antigen-binding domain. In some of these embodiments, the magnetic nanoparticle comprises an antibody or an antigen-binding domain. In certain embodiments, the antibody or an antigen-binding fragment further comprises biotin.

1.4.3 Exosome Target

In various embodiments, the biological target is an exosome. In some of these embodiments, the magnetic nanoparticle comprises an antibody against an exosome-specific surface marker, such as CD63, CD81, or CD9. In some of these embodiments, the magnetic nanoparticle comprises a polynucleotide that can hybridize with an exosome specific polynucleotide.

1.4.4 Cell Target

In some embodiments, the biological target is a cell. In various embodiments, the target cell is a T-cell, a B-cell, a dendritic cell, a natural killer (NK) cell, a monocyte, a macrophage, a granulocyte, a myeloid cell, an cytokine-producing cell, an endothelial cell, a tumor cell, a cancer stem cell, a hematopoietic stem cell, a mesenchymal stem cell, an embryonic stem (ES) cell, an induced pluripotent stem (iPS) cell, a neural cell, or a cardiac cell. In certain embodiments, the magnetic nanoparticle comprises an antibody against a surface marker of a specific cell type.

In some embodiments, the biological target is a tumor cell. In certain embodiments, the biological target is a circulating tumor cell (CTC).

1.4.5 Method of Enriching cfDNA

Also provided herein are methods of enriching circulating free DNA (cfDNA) from a body fluid. The methods comprise: a) providing a sample of body fluid containing or suspected of containing cfDNA; b) adding to the sample a solution of dispersed magnetic nanoparticles capable of binding the cfDNA under conditions that permit a complex to form between the magnetic nanoparticle and the cfDNA; c) separating the complex from the body fluid by application of an external magnetic field; and d) recovering the cfDNA from the magnetic nanoparticles.

In various embodiments, the body fluid is blood, serum, plasma, saliva, cerebrospinal fluid, urine, semen, or ascites. In specific embodiments, the body fluid is blood, serum, or plasma.

In some embodiments, the method further comprises the earlier step of pretreating the body fluid to remove cells. In some embodiments, the method further comprises a lysis step to effect the release of the cfDNA. In some embodiments, the method further comprised a protein precipitation step to remove protein. In certain embodiments, the method further comprises the step of washing the complex formed between the magnetic nanoparticle and the cfDNA to remove contaminants. In some embodiments, the complex is washed with one or more washing buffer. In other embodiments, the method further comprises the step of eluting the cfDNA off the magnetic nanoparticles to obtain an enriched sample of cfDNA.

The enriched cfDNA can be analyzed by various techniques, such as NGS (Next Generation Sequencing), targeted sequencing, PCR (Polymerase Chain Reaction), real time PCR, digital PCR, cold-PCR (co-amplification at lower denaturation temperature-PCR), BEAMing (Beads, Emulsification, Amplification, and Magnetics), MAP (MIDI-Activated Pyrophosphorolysis), and mass spectrometry genotyping assay-mutant-enriched PCR. In certain embodiments, the method further comprises analyzing the entirety or a portion of the enriched cfDNA by NGS or targeted sequencing.

In some embodiments, the body fluid is collected from a patient with, or suspected of having, cancer, such as breast cancer, prostate cancer, skin cancer, colon cancer, lung cancer, liver cancer, leukemia, lymphoma, brain cancer, etc. In some of these embodiments, the cfDNA comprises cancer specific DNA. In some embodiments, the body fluid is collected from a patient with, or suspecting of having, an infectious disease, such as influenza, hepatitis A, hepatitis B, hepatitis C, HIV/AIDS, chickenpox, smallpox, etc. In some of these embodiments, the cfDNA comprises virus specific DNA. In some embodiments, the body fluid is collected from a pregnant woman. In some of these embodiments, the cfDNA comprises fetal DNA.

1.4.6 Method of Enriching cfRNA

Also provided herein are methods of enriching circulating free RNA (cfRNA) from a body fluid. The methods comprise: a) providing a sample of body fluid containing or suspected of containing cfRNA; b) adding to the sample a solution of dispersed magnetic nanoparticles capable of binding the cfRNA under conditions that permit a complex to form between the magnetic nanoparticle and the cfRNA; c) separating the complex from the body fluid by application of an external magnetic field; and d) recovering the cfRNA from the magnetic nanoparticles.

In various embodiments, the body fluid is blood, serum, plasma, saliva, cerebrospinal fluid, urine, semen, or ascites. In specific embodiments, the body fluid is blood, serum, or plasma.

In some embodiments, the method further comprises the earlier step of pretreating the body fluid to remove cells. In some embodiments, the method further comprises a lysis step to effect the release of the cfRNA. In some embodiments, the method further comprised a protein precipitation step to remove protein. In certain embodiments, the method further comprises the step of washing the complex formed between the magnetic nanoparticle and the cfRNA to remove contaminants. In some embodiments, the complex is washed with one or more washing buffer. In other embodiments, the method further comprises the step of eluting the cfRNA off the magnetic nanoparticles to obtain an enriched sample of cfRNA. In certain embodiments, the method further comprises a DNase treatment step to remove the contaminant DNA.

The enriched cfRNA can be analyzed by various techniques, such as NGS (Next Generation Sequencing), targeted sequencing, RT-PCR (Reverse Transcription Polymerase Chain Reaction), real time PCR, digital PCR, cold-PCR (co-amplification at lower denaturation temperature-PCR), BEAMing (Beads, Emulsification, Amplification, and Magnetics), MAP (MIDI-Activated Pyrophosphorolysis), and mass spectrometry genotyping assay-mutant-enriched PCR. In certain embodiments, the method further comprises analyzing the entirety or a portion of the enriched cfRNA by NGS or targeted sequencing.

In some embodiments, the body fluid is collected from a patient with, or suspected of having, cancer, such as breast cancer, prostate cancer, skin cancer, colon cancer, lung cancer, liver cancer, leukemia, lymphoma, brain cancer, etc. In some of these embodiments, the cfRNA comprises cancer specific RNA. In some embodiments, the body fluid is collected from a patient with, or suspecting of having, an infectious disease, such as influenza, hepatitis A, hepatitis B, hepatitis C, HIV/AIDS, chickenpox, smallpox, etc. In some of these embodiments, the cfRNA comprises virus specific RNA. In some embodiments, the body fluid is collected from a pregnant woman. In some of these embodiments, the cfRNA comprises fetal RNA.

1.4.7 Method of Enriching Circulating Tumor Cell (CTC)

Also provided herein are methods of enriching circulating tumor cell (CTC) from a body fluid. The methods comprise: a) providing a sample of body fluid containing the circulating tumor cell; b) adding to the sample a solution of dispersed magnetic nanoparticles capable of binding the circulating tumor cell under conditions that permit a complex to form between the magnetic nanoparticle and the circulating tumor cell; and c) separating the complex from the body fluid by application of an external magnetic field.

In various embodiments, the body fluid is blood, serum, plasma, saliva, cerebrospinal fluid, urine, semen, or ascites. In specific embodiments, the body fluid is blood, serum, or plasma.

In some embodiments, the method further comprises the earlier step of pretreating the body fluid to enrich cells. In certain embodiments, the method further comprises the step of washing the complex formed between the magnetic nanoparticle and the circulating tumor cell to remove contaminants. In some embodiments, the complex is washed with one or more washing buffer. In other embodiments, the method further comprises the step of recovering the circulating tumor cell from the magnetic nanoparticles by eluting the circulating tumor cell off the magnetic nanoparticles.

In some embodiments, the method further comprises analyzing the enriched circulating tumor cell. In certain embodiments, the method further comprises analyzing the size and shape of the circulating tumor cell. In certain embodiments, the method further comprises analyzing the surface biomarker of the circulating tumor cell. In certain embodiments, the method further comprises sequencing the DNA/RNA of the circulating tumor cell. The enriched circulating tumor cell can be analyzed by various techniques, such as immunostaining, flow cytometry, fluorescence microscopy, microscopy, NGS (Next Generation Sequencing), targeted sequencing, PCR (Polymerase Chain Reaction), real time PCR, digital PCR, cold-PCR (co-amplification at lower denaturation temperature-PCR), BEAMing (Beads, Emulsification, Amplification, and Magnetics), MAP (MIDI-Activated Pyrophosphorolysis), and mass spectrometry genotyping assay-mutant-enriched PCR.

In some embodiments, the body fluid is collected from a patient with, or suspected of having, cancer, such as breast cancer, prostate cancer, skin cancer, colon cancer, lung cancer, liver cancer, leukemia, lymphoma, brain cancer, etc.

1.5 Method of Preparing Magnetic Nanoparticles

Also disclosed herein are methods of preparing a magnetic nanoparticle. The method comprises: a) making a dispersion comprising a metal salt, an organic solvent, and a capping reagent; b) heating the dispersion; c) isolating magnetic cores from the dispersion; d) adding a silicon or titanium organic compound to the magnetic cores; e) hydrolyzing at least some of the silicon or titanium organic compound; and f) crosslinking the hydrolyzed silicon or titanium organic compound on the surface of the magnetic cores.

1.5.1 Method of Preparing the Magnetic Core

In various embodiments, the method of preparing the magnetic core comprises: making a dispersion comprising a metal salt, an organic solvent, and a capping reagent; heating the dispersion; and isolating magnetic cores from the dispersion.

In some embodiments, the metal salt comprises an iron salt, such as Iron (III) citrate, or its dichlorotetrakis, bromide, fluoride, iodide, molybate, oxalate, perchlorate, phosphate, acetate, chloride, sulfate, nitrate, pyrophosphate, tetrafluoroborate, and hexacyano complexed salt. In some embodiments, the metal salt further comprises a salt of a second metal, such as a metal citrate, or its dichlorotetrakis, bromide, fluoride, iodide, molybate, oxalate, perchlorate, phosphate, acetate, chloride, sulfate, nitrate, pyrophosphate, tetrafluoroborate, and hexacyano complexed salt. In certain embodiments, the second metal is Mn, Ca, Co, Zn, Cu, Mg, Ba, Ni, or Cr.

In various embodiments, the organic solvent is ethylene glycol, polyethylene glycol, ethanol, methanol, propanol, isopropanol, propylene glycol, or polypropylene glycol. In some embodiments, the capping agent is polyethylene glycol, polypropylene glycol, acetate ions, citrate ions, formate ions, propionic ions, or succinate ions.

In various embodiments, the dispersion further comprises a first surfactant. Non-limiting examples of first surfactant include ionic surfactants, such as alkylbenzene sulfonate, fatty acid soap, lauryl sulfate, di-alkyl sulfosuccinate, lignosulfonate, fatty amine salt, and quaternary ammonium, and non-ionic surfactant, such as polyoxyethylene fatty alcohol ether, polyoxyethylene alkylphenyl ether, and polyoxyethylene-polyoxypropylene block copolymer.

In various embodiments, the dispersion is heated to 180-240° C., such as about 180° C., about 190° C., about 200° C., about 210° C., about 220° C., about 230° C., or about 240° C. In various embodiments, the dispersion is heated for 4-80 hours, such as about 4 hours, about 10 hours, about 20 hours, about 30 hours, about 40 hours, about 50 hours, about 60 hours, about 70 hours, or about 80 hours. In certain embodiments, the dispersion is heated in an autoclave. In specific embodiments, the dispersion is heated in an autoclave at about 200° C. for about 60 hours. In specific embodiments, the dispersion is heated in an autoclave at about 220° C. for about 60 hours. In specific embodiments, the dispersion is heated in an autoclave at about 240° C. for about 60 hours. In specific embodiments, the dispersion is heated in an autoclave at about 200° C. for about 40 hours. In specific embodiments, the dispersion is heated in an autoclave at about 220° C. for about 40 hours. In specific embodiments, the dispersion is heated in an autoclave at about 240° C. for about 40 hours. In specific embodiments, the dispersion is heated in an autoclave at about 200° C. for about 20 hours. In specific embodiments, the dispersion is heated in an autoclave at about 220° C. for about 20 hours. In specific embodiments, the dispersion is heated in an autoclave at about 240° C. for about 20 hours.

In various embodiments, the magnetic cores are isolated from the dispersion. In some embodiments, the isolation comprises cooling, washing, and drying the magnetic cores. In certain embodiments, the magnetic cores are cooled to room temperature. In certain embodiments, the magnetic cores are washed with ethanol. In certain embodiments, the magnetic cores are dried for 12 to 72 hours, such as about 24 hours, about 36 hours, about 48 hours, or about 60 hours.

1.5.2 Method of Preparing the Outer Shell

In some embodiments, the method of preparing the outer shell comprises: adding a silicon or titanium organic compound to the magnetic cores; hydrolyzing at least some of the silicon or titanium organic compound; and crosslinking the hydrolyzed silicon or titanium organic compound on the surface of the magnetic cores. In various embodiments, the silicon organic compound is Allyltriethoxysilane, Allyltrimethoxysilane, [3-(2-Aminoethylamino)propyl]trimethoxysilane, 3-Aminopropyl(diethoxy)methylsilane, (3-Aminopropyl)triethoxysilane, Azidotrimethylsilane, 1,2-Bis(trichlorosilyl)ethane, 1,2-Bis(triethoxysilyl)ethane, 1,2-Bis(trimethoxysilyl)ethane, (3-Bromopropyl)trimethoxysilane, Butyltrichlorosilane, Chloromethyl(methyl)dimethoxysilane, Chloromethyltrimethoxysilane, 3-Cyanopropyltriethoxysilane, Diethoxydimethylsilane, Dodecyltriethoxysilane, Hexadecyltrimethoxysilane, Methoxytrimethylsilane, Octamethylcyclotetrasiloxane, n-Propyltriethoxysilane, Trimethoxyphenylsilane, Tetraethyl orthosilicate, Tetramethyl orthosilicate, or Vinyltrimethoxysilane. In various embodiments, the titanium organic compound is Chlorotriisopropoxytitanium(IV), Dichlorobis(indenyl)titanium(IV), Tetrakis(diethylamido)titanium(IV), Titanium(IV) butoxide, Titanium(IV) tert-butoxide, Titanium(III) chloride tetrahydrofuran complex, Titanium(IV) ethoxide, Titanium(IV) 2-ethylhexyloxide, Titanium(IV) isopropoxide, Titanium(IV) methoxide, Titanium(IV) oxyacetylacetonate, Titanium(IV) (triethanolaminato)isopropoxide, or Trichloro(pentamethylcyclopentadienyl)titanium(IV). In some embodiments, the method further comprises dispersing the magnetic core in a solvent.

In some embodiments, the method of preparing the outer shell comprises: coating the magnetic core with a polymer layer by chemical bonding, or coating the magnetic core with a monomer layer by chemical bonding and performing a polymerization reaction on the magnetic core. The polymer can be highly cross-linked during the polymerization reaction. In some embodiments, the polymer is polyethylene glycol, polyacrylic acid, polyacrylamide, polyvinyl alcohol, poly-methyl methacrylate, polystyrene, poly-4-vinylphenol, polyester, polyimide, polyethylene, polypropylene, polyethylene vinyl acetate, polyacrylates, or polysaccharide.

In certain embodiments, the method of preparing the outer shell further comprises: dispersing the magnetic cores in solution comprising a second surfactant before adding the silicon organic compound, titanium organic compound, polymer, or monomer to the magnetic cores. In some of these embodiments, a first surfactant was present during preparation of the core. In some of these embodiments, a first surfactant was not present during preparation of the core.

In some embodiments, the second surfactant self-assembles on the magnetic core. In some of these embodiments, the method further comprises removing the self-assembled surfactant by ion exchange. In various embodiments, the magnetic nanoparticle comprises a mesoporous outer shell.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1: Preparation of the Magnetic Nanoparticle

Making the Magnetic Core:

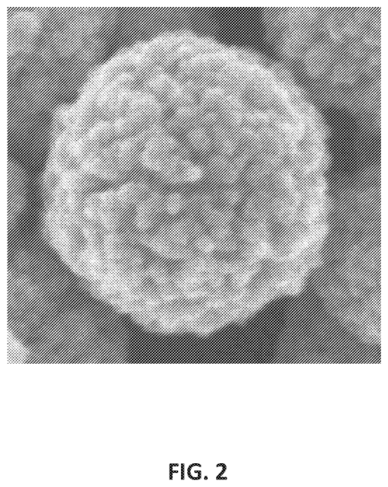

1 g Iron(III) citrate was dissolved in propylene glycol (100 mL) to form a clear solution, followed by the addition of 5 g polyoxyethylene-polyoxypropylene block copolymer. The mixture was stirred vigorously for 60 min and then transferred to a sealed stainless steel autoclave. The autoclave was heated to 220° C. and maintained for 40 hours, and then allowed to cool to room temperature. The resulting magnetic cores were washed several times with ethanol and dried at room temperature for 48 hours. An example of the magnetic core generated is shown in FIG. 2.

Making the Outer Shell:

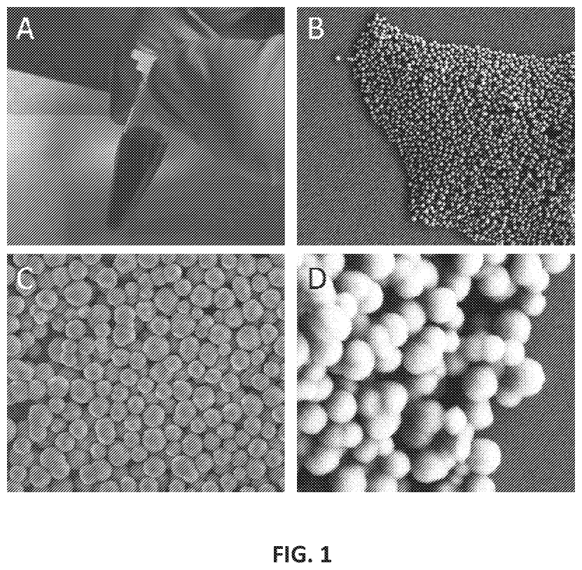

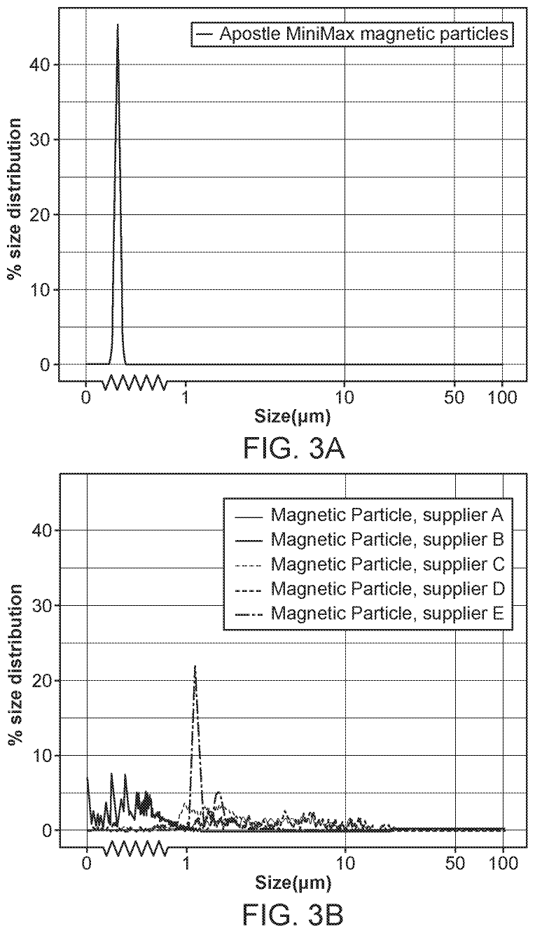

50 mg magnetic core were dispersed in the solvents (20 ml ethanol, 20 ml isopropanol, 20 ml deionized water, 3 ml 1 M Tetramethylammonium hydroxide solution) followed by the dropwise addition of 0.5 mL Tetramethyl orthosilicate. The mixture was mechanically stirred at room temperature for 3 h. The magnetic nanoparticle was washed with deionized water 3 times, and dried at 60° C. for 6 hours. The magnetic nanoparticles generated are shown in FIG. 1A-1D. As shown in FIG. 3A, the size distribution of the magnetic nanoparticle was measured by dynamic light scattering particle size analyzer. Compared to magnetic beads from other suppliers in FIG. 3B, the magnetic nanoparticle has a more uniform size distribution. Doublet is not detectable with the magnetic nanoparticle.

Making the Mesoporous Shell:

50 mg magnetic core were dispersed in the solvents (20 ml ethanol, 20 ml isopropanol, 20 ml deionized water, 3 ml 1M Tetramethylammonium hydroxide solution, 1 ml Polyoxyethylene (20) oleyl ether), stirred for 1 hour, followed by the dropwise addition of 0.5 mL Tetramethyl orthosilicate. The mixture was mechanically stirred at room temperature for 3 h. The product was washed with deionized water 3 times, and dried at 60° C. for 6 hours. The dried product was re-dispersed in 100 mL deionized water solution containing 1 g Ammonium Nitrate and reflux at 60° C. for 24 hours. The final product, the mesoporous magnetic nanoparticle, was washed with deionized water 3 times and dried at 60° C. for 6 hours.

### Example 2: Isolation of DNA ladder from TE Buffer

A sample of DNA ladder (50-3000 bp, Sigma-Aldrich, Cat #57025) was spiked into TE buffer. The final concentration of the DNA ladder was 100 ng/ml. The DNA sample was incubated with the magnetic nanoparticle prepared as described in Example 1 in the presence of 2-3 M guanidine thiocyanate. The mixture with magnetic nanoparticle suspension was put on a magnet for a few minutes, or until the solution was clear and magnetic nanoparticles were pelleted. The supernatant was then removed. The separated magnetic nanoparticles were washed with 2-3 M guanidine thiocyanate solution and 80% ethanol solution. The DNA ladder was eluted from the magnetic nanoparticles with TE buffer. The isolated DNA was characterized using an Agilent 2100 bioanalyzer. A commercially available magnetic bead from a major supplier was used for the DNA purification in parallel.

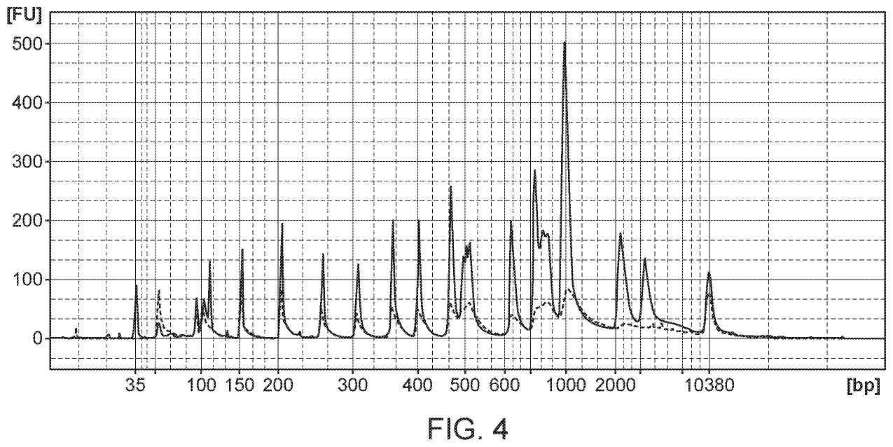

As shown in FIG. 4, the magnetic nanoparticle prepared as in Example 1 (solid line) was more efficient than the commercially available magnetic bead (dotted line) in isolating DNA of different sizes from TE buffer.

### Example 3: Isolation of DNA Ladder from Serum

DNA ladder (50-3000 bp, Sigma-Aldrich, Cat #57025) was spiked into fetal bovine serum (FBS) (Thermo Fisher Scientific, Cat #26140079). The final concentration of the DNA ladder was 100 ng/ml. The DNA sample was incubated with magnetic nanoparticles manufactured as described in Example 1 in the presence of 2-3 M guanidine thiocyanate. The mixture with magnetic nanoparticle suspension was put on a magnet for a few minutes, or until the solution was clear and magnetic nanoparticles were pelleted. The supernatant was then removed. The separated magnetic nanoparticles were washed with 2-3 M guanidine thiocyanate solution and 80% ethanol solution. The DNA ladder was eluted from the magnetic nanoparticles with TE buffer. The isolated DNA was characterized using an Agilent 2100 bioanalyzer. A commercially available magnetic bead from a major supplier was used for the DNA purification in parallel. The size distribution of the original DNA ladder was also analyzed.

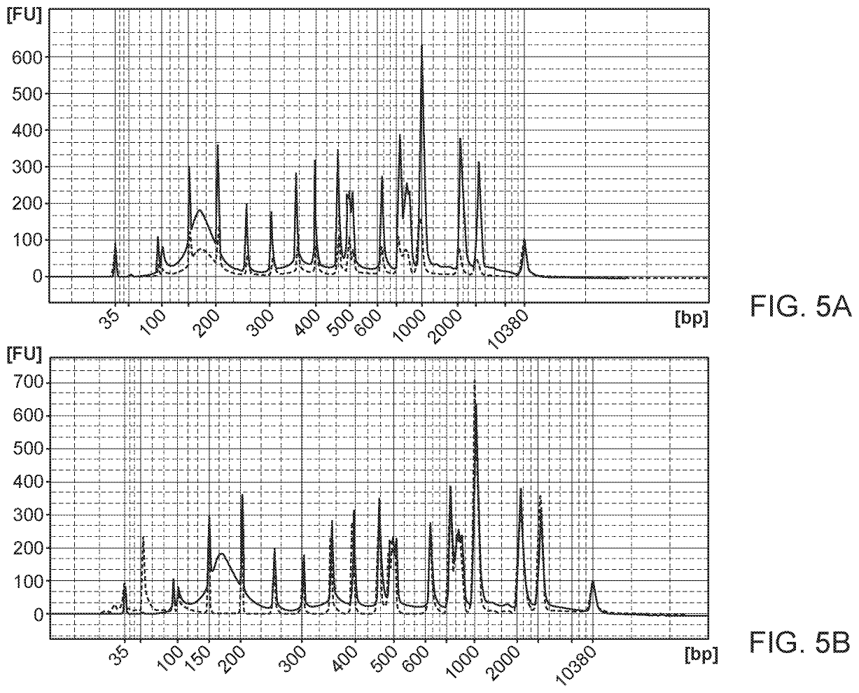

As shown in FIG. 5A, the magnetic nanoparticles prepared as in Example 1 (solid line) were more efficient than the commercially available magnetic bead (dotted line) in isolating DNA of different sizes from FBS. Additionally, by comparing the size of distribution of the DNA ladder enriched with the magnetic nanoparticles of Example 1 (solid line) with the size distribution of the original DNA ladder (dotted line), FIG. 5B shows that DNA enriched using the Example 1 magnetic nanoparticles had a close to 100% recovery efficiency. Note that the peak between 150-200 bp represents DNA molecules from FBS, not the DNA ladder.

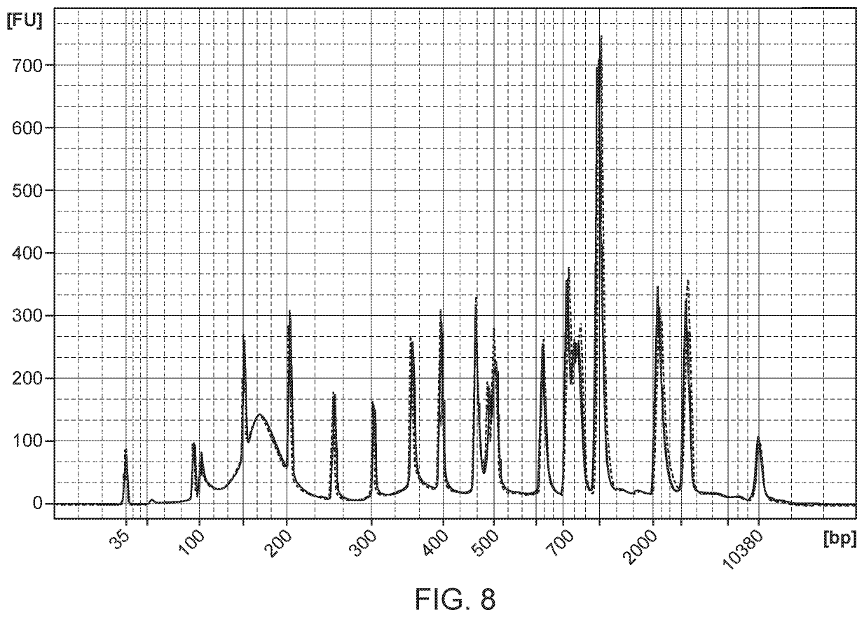

Three different batched of the magnetic nanoparticles prepared as in Example 1 were used to isolated DNA from FBS. The isolated DNA was characterized using an Agilent 2100 bioanalyzer. As shown in FIG. 8, different batches of magnetic nanoparticles demonstrated consistent DNA isolation results.

### Example 4: Isolation of DNA from Human Plasma

4 mL human plasma was obtained from a healthy donor and was pre-treated with lysis buffer containing surfactant and proteinase. The treated human plasma was then incubated with 1-3 mg magnetic nanoparticles in the presence of 2-3 M guanidine thiocyanate. The mixture with magnetic nanoparticle suspension was put on a magnet for a few minutes, or until the solution was clear and magnetic nanoparticles were pelleted. The supernatant was then removed. The separated magnetic nanoparticles were washed with 2-3 M guanidine thiocyanate solution and 80% ethanol solution. The DNA from human plasma was eluted from the magnetic nanoparticles with TE buffer. The isolated DNA was characterized using an Agilent 2100 bioanalyzer. A commercially available magnetic bead from a major supplier was used for the DNA purification in parallel.

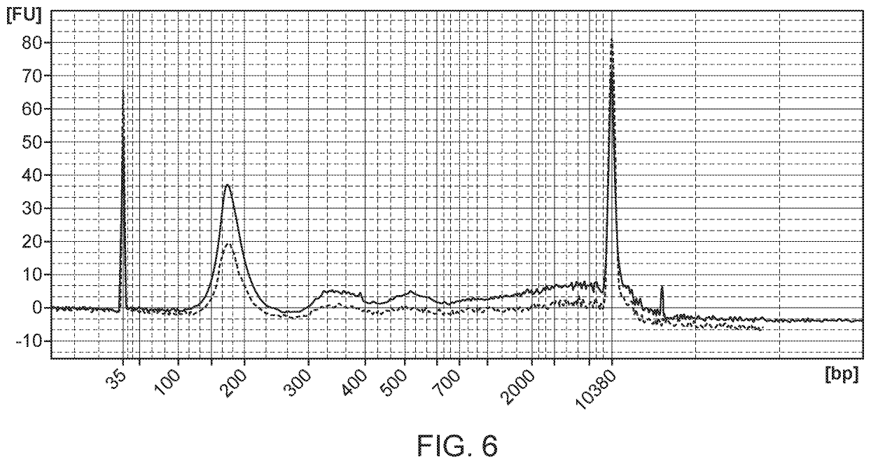

As shown in FIG. 6, the magnetic nanoparticle (solid line) is more efficient than the commercially available magnetic bead (dotted line) in isolating DNA from human plasma.

### Example 5: Isolation of DNA from Human Urine

20 mL human urine was obtained from a healthy donor and was pre-treated with lysis buffer containing surfactant and proteinase. The treated human urine was then incubated with 5-10 mg magnetic nanoparticles in the presence of 2-3 M guanidine thiocyanate. The mixture with magnetic nanoparticle suspension was put on a magnet for a few minutes, or until the solution was clear and magnetic nanoparticles were pelleted. The supernatant was then removed. The separated magnetic nanoparticles were washed with 2-3 M guanidine thiocyanate solution and 80% ethanol solution. The DNA from human urine was eluted from the magnetic nanoparticles with TE buffer. The isolated DNA was characterized using an Agilent 2100 bioanalyzer. A commercially available magnetic bead from a major supplier was used for the DNA purification in parallel.

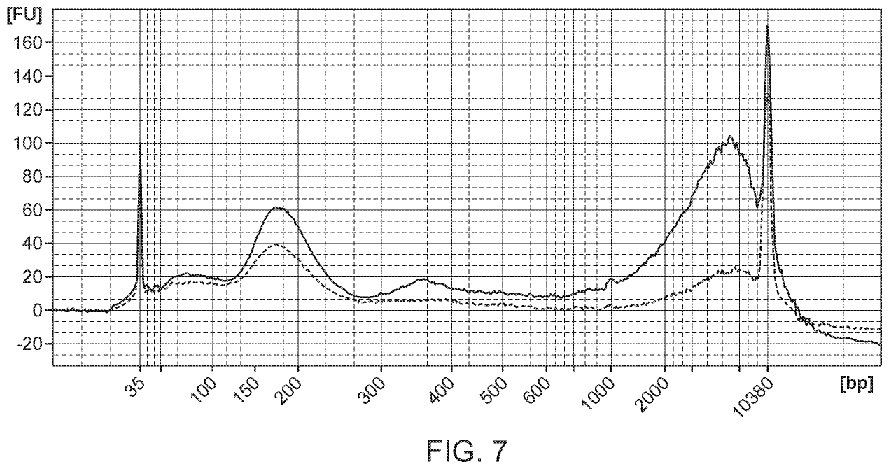

As shown in FIG. 7, the magnetic nanoparticle (solid line) is more efficient than the commercially available magnetic bead (dotted line) in isolating DNA from human urine.

### Example 6: Isolation of High Quality DNA for Mutant Detection

20 μL DNA fragment containing the EGFR c.2573T>G (L858R) mutation (synthetic, ˜170 bp) at concentration of 1 ng/μL, 0.1 ng/μL, 0.01 ng/μL, or 0.001 ng/μL, was spiked in 1 mL TE buffer or FBS. The mutated DNA fragment was isolated with the magnetic nanoparticles. The procedure of DNA isolation from TE buffer and serum with the magnetic nanoparticles is described in Examples 2 and 3. The final elution volume was 20 μL. qPCR assay was performed using 1 μL of the isolated DNA from TE buffer or serum. The qPCR results were compared with 1 μL of the original mutated DNA solution at 1 ng/μL, 0.1 ng/μL, 0.01 ng/μL, or 0.001 ng/μL.

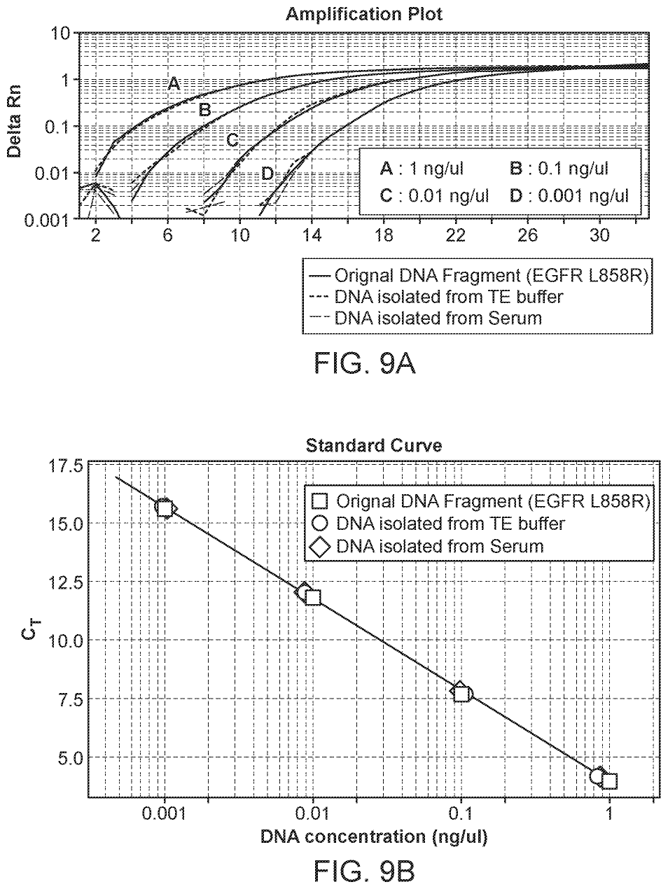

The qPCR amplification plot (FIG. 9A) shows that the amplification curves of the DNA fragment isolated from TE buffer or serum overlap with the amplification curves of the original DNA solution at all tested concentrations. The qPCR standard curves of DNA fragment isolated from TE buffer and serum also overlap with the standard curve of the original DNA solution (FIG. 9B). These results indicate the magnetic nanoparticle described herein isolates DNA efficiently (>90%) and with high quality. The isolated DNA is suitable for downstream applications, such as qPCR.

### Example 7: Isolation of Small DNA

DNA ladder (50-3000 bp, Sigma-Aldrich, Cat #57025) was spiked in fetal bovine serum (FBS). The final concentration of the DNA ladder was 100 ng/ml. The protein in the FBS was precipitated using a protein precipitation solution comprising zinc chloride. The supernatant was incubated with magnetic nanoparticles manufactured as described in Example 1 in the presence of 2-3 M guanidine thiocyanate. The mixture with magnetic nanoparticle suspension was put on a magnet for a few minutes, or until the solution was clear and magnetic nanoparticles were pelleted. The supernatant was then removed. The separated magnetic nanoparticles were washed with 2-3 M guanidine thiocyanate solution and 80% ethanol solution. The DNA ladder was eluted from the magnetic nanoparticles with TE buffer. The isolated DNA was characterized using an Agilent 2100 bioanalyzer. The size distribution of the original DNA ladder was also analyzed.

cfDNA Reference Standard (Horizon Discovery Ltd, Cat # HD780) was spiked in TE buffer. The sample was incubated with magnetic nanoparticles manufactured as described in Example 1 in the presence of 2-3 M guanidine thiocyanate. The mixture with magnetic nanoparticle suspension was put on a magnet for a few minutes, or until the solution was clear and magnetic nanoparticles were pelleted. The supernatant was then removed. The separated magnetic nanoparticles were washed with 2-3 M guanidine thiocyanate solution and 80% ethanol solution. The cfDNA sample was eluted from the magnetic nanoparticles with TE buffer. The isolated cfDNA was characterized using an Agilent 2100 bioanalyzer. A commercially available magnetic bead from a major supplier was used for the cfDNA purification in parallel. The size distribution of the original cfDNA Reference Standard was also analyzed.

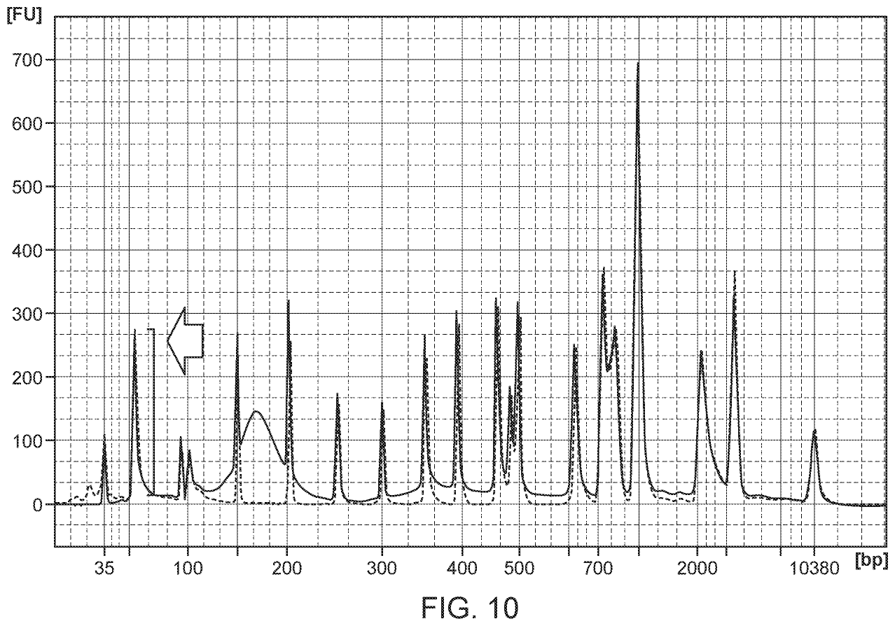

As shown in FIG. 10, by comparing to the original DNA ladder (dotted line), DNA enriched using the Example 1 magnetic nanoparticles (solid line) had a greater than 95% recovery efficiency, including for small DNA fragments of about 50 bp. Note that the peak between 150-200 bp represents DNA molecules from FBS, not the DNA ladder.

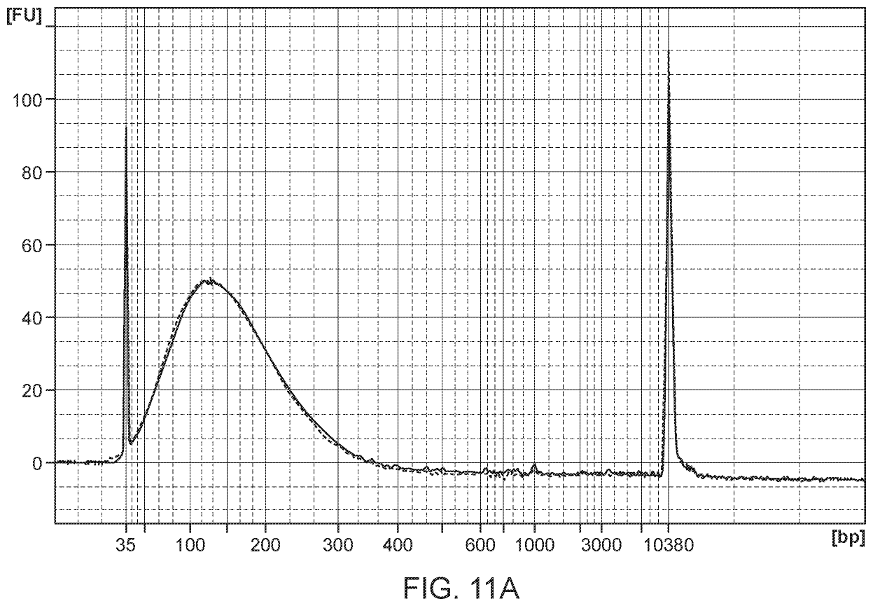

As shown in FIG. 11A, by comparing to the original cfDNA Reference Standard (dotted line), the magnetic nanoparticles (solid line) had a greater than 95% recovery efficiency for the cfDNA Reference Standard, which include a significant portion of DNA fragments of less than 100 bp. FIGS. 11B and 11C show that the magnetic nanoparticle (solid line) was more efficient than the commercially available magnetic bead (dotted line) in isolating DNAs of 35-200 bp from TE buffer.

### Example 8: Isolation of Single-Stranded DNA

Single-stranded DNA plays an important role in various bioprocesses. Compared to double-stranded DNA, single-stranded DNA is more difficult to isolate from body fluid, due to its smaller size.

30 μL single-stranded DNA fragment containing the EGFR c.2573T>G (L858R) mutation (synthetic, ˜170 nt) at concentration of 0.001 ng/μL was spiked in 2 mL FBS, resulting a final single-stranded DNA concentration of 0.015 pg/μl. The single-stranded DNA fragment was isolated with the magnetic nanoparticles described herein. The procedure of single-stranded DNA isolation with the magnetic nanoparticles is the same as the procedure described in Example 7. The final elution volume was 30 μL. qPCR assay was performed using the isolated single-stranded DNA. A commercially available magnetic bead from a major supplier was used for the DNA purification in parallel. The qPCR results were compared to the original single-stranded DNA solution.

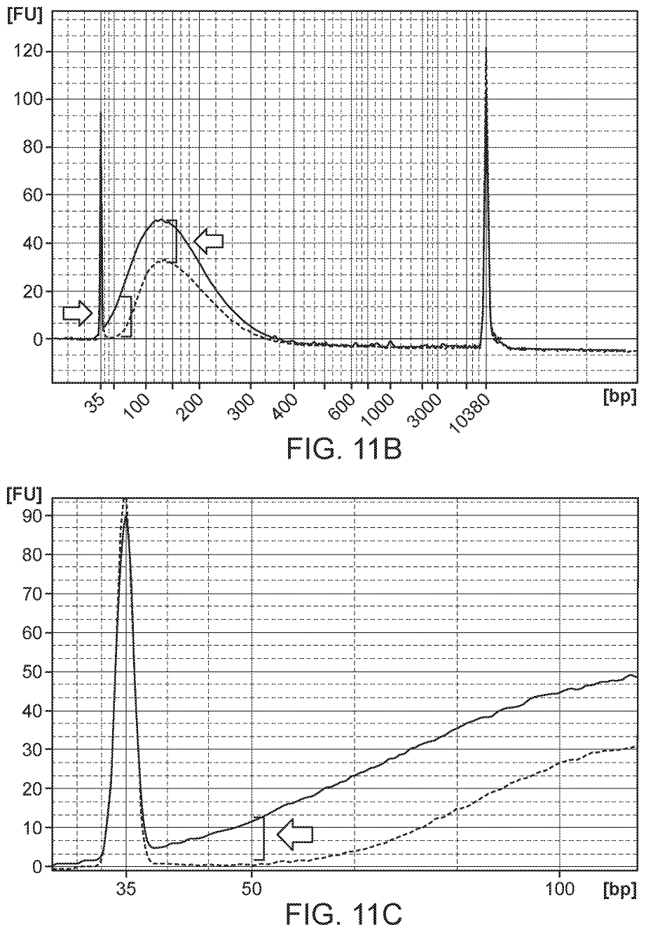

As shown in FIGS. 12A and 12B, the single-stranded DNA isolated using the Example 1 magnetic nanoparticles had a greater than 95% recovery efficiency as compared to the original input reference, while the commercial magnetic bead from a major supplier only had an about 10% recovery efficiency.

### Example 9: Isolation of RNA Ladder from Serum or Plasma

cfRNAs are usually present in body fluids as small RNA fragments (<1000 nt) and even smaller cell-free miRNAs (˜20 nt). Although the concentration of cfRNAs is low, they are important biomarkers for cancer and other diseases.

RNA ladder (100-1000 nt,) was spiked in human plasma. The protein in the serum was precipitated using a protein precipitation solution comprising zinc chloride. The supernatant was incubated with magnetic nanoparticles manufactured as described in Example 1 in the presence of 2-3 M guanidine thiocyanate. The mixture with magnetic nanoparticle suspension was put on a magnet for a few minutes, or until the solution was clear and magnetic nanoparticles were pelleted. The supernatant was then removed. The separated magnetic nanoparticles were washed with 2-3 M guanidine thiocyanate solution and 80% ethanol solution. The RNA ladder was eluted from the magnetic nanoparticles with TE buffer. The isolated RNA was characterized using an Agilent 2100 bioanalyzer. The original RNA ladder was also analyzed.

Small RNA ladder (17-150 nt,) was spiked in human plasma. The protein in the plasma was precipitated using a protein precipitation solution. The supernatant was incubated with magnetic nanoparticles manufactured as described in Example 1 in the presence of 2-3 M guanidine thiocyanate. The mixture with magnetic nanoparticle suspension was put on a magnet for a few minutes, or until the solution was clear and magnetic nanoparticles were pelleted. The supernatant was then removed. The separated magnetic nanoparticles were washed with 2-3 M guanidine thiocyanate solution and 80% ethanol solution. The small RNA ladder was eluted from the magnetic nanoparticles with TE buffer. The isolated RNA was characterized using an Agilent 2100 bioanalyzer. The original small RNA ladder was also analyzed.

For certain downstream applications, such as qPCR, the RNA was treated with DNase on the magnetic nanoparticles after the washing step and before the elution step.

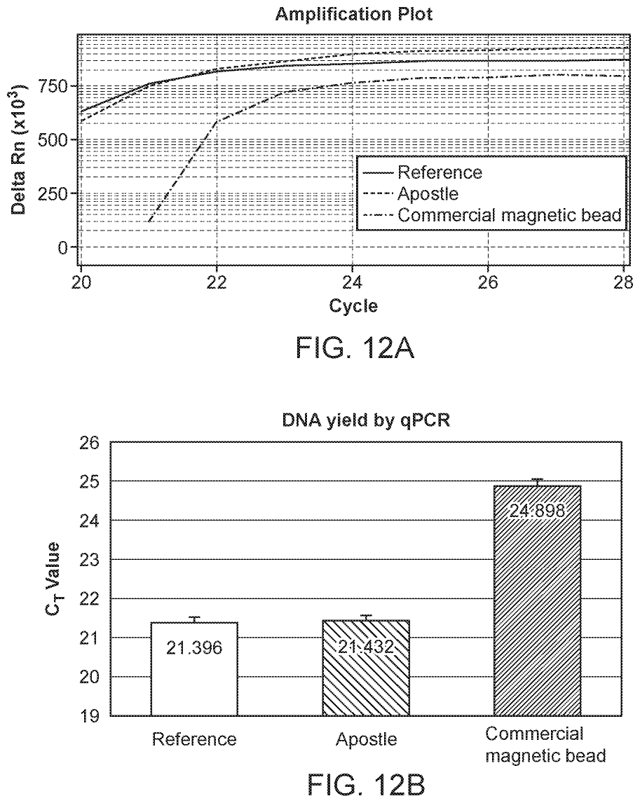

As shown in FIG. 13, comparing to the original RNA ladder (dotted line), the magnetic nanoparticles (solid line) had a greater than 90% recovery efficiency for RNA fragments between 100 nt and 1000 nt.

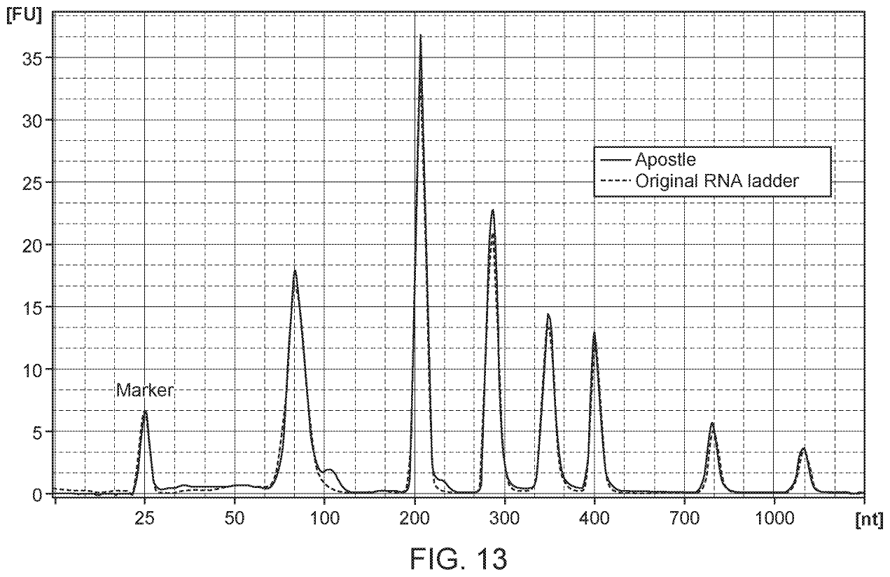

As shown in FIG. 14, comparing to the original small RNA ladder (dotted line), the magnetic nanoparticles (solid line) had a greater than 90% recovery efficiency for small RNA fragments between 17 nt and 150 nt.

### Example 10: Isolation of Exogenous RNA from Blood

Blood was collected in three different blood collection tubes (BCTs), K₃EDTA BCT, cfRNA BCT vendor 1, and cfRNA BCT vendor 2, respectively and processed to obtain cell-free plasma. cfRNA BCT vendor 1 and cfRNA BCT vendor 2 are specialized blood collection tubes which prevent RNA degradation during storage. A synthetic RNA mimic cel-miR-39 was spiked in the plasma. The RNA mimic was isolated with the magnetic nanoparticles. The procedure of RNA isolation from plasma with the magnetic nanoparticles is the same as the procedure described in Example 9. RT-qPCR assay was performed using the isolated RNA mimic. A commercially available column-based RNA isolation product from a major supplier was used for the RNA purification in parallel.

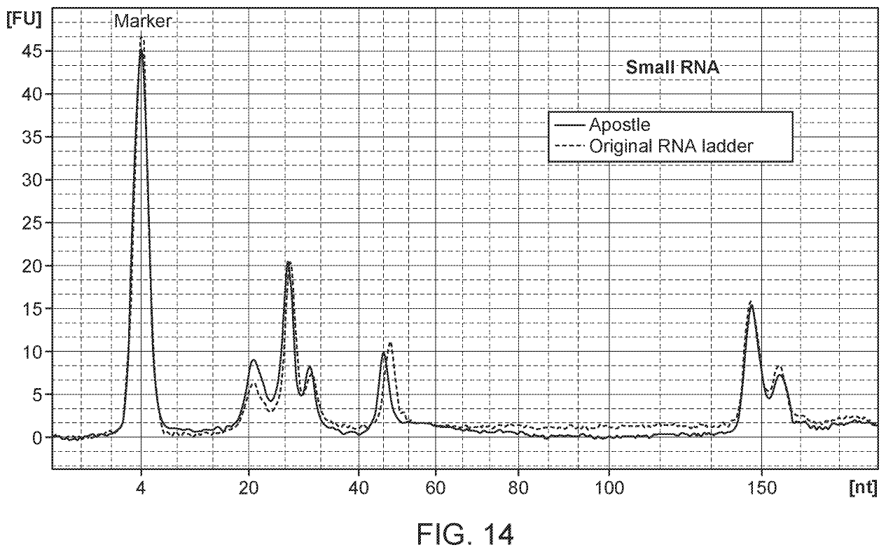

As shown in FIGS. 15A, 15B, and 15C, the RNA recovery rate of the magnetic nanoparticle was 1.1, 9, and 2800 times of the RNA recovery rate of the column-based RNA isolation product for blood collected in K₃EDTA BCT, cfRNA BCT vendor 1, and cfRNA BCT vendor 2, respectively. These results indicate that the magnetic nanoparticle is more efficient than the column-based RNA isolation product in isolating exogenous RNA from blood collected in various blood collection tubes, especially from blood collected in the specialized BCTs that prevent RNA degradation during storage.

### Example 11: Isolation of Endogenous RNA from Plasma

Blood was collected in three different blood collection tubes (BCTs), K₃EDTA BCT, cfRNA BCT vendor 1, and cfRNA BCT vendor 2, respectively and processed to obtain cell-free plasma. cfRNA BCT vendor 1 and cfRNA BCT vendor 2 are specialized blood collection tubes which prevent RNA degradation during storage. The endogenous cfRNA was isolated from 200 uL plasma with the magnetic nanoparticles as described in Example 9. The isolated cfRNAs of beta-globin, miR-21, miR-U6, and miR-15a were measured by qPCR. A commercially available column-based RNA isolation product from a major supplier was used for the RNA purification in parallel.

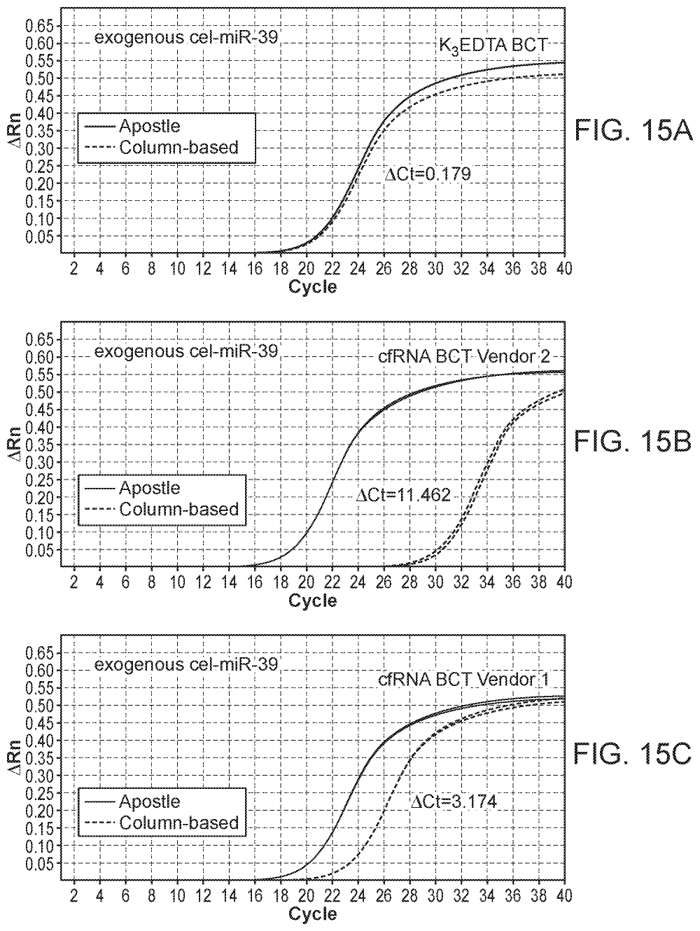

FIGS. 16A-D show that for blood collected in K₃EDTA BCT, the RNA recovery rate of the magnetic nanoparticle was 2.1, 1.1, 2.4, and 1.3 times of the RNA recovery rate of the column-based RNA isolation product for the cfRNA of beta-globin, miR-21, U6, and miR-15a, respectively.

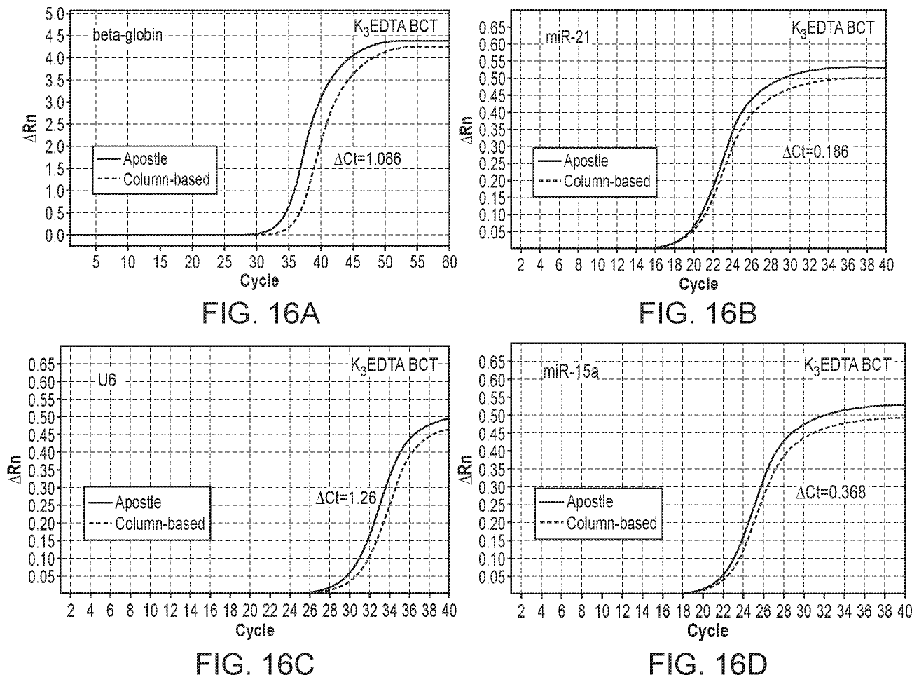

FIGS. 17A-D show that for blood collected in cfRNA BCT vendor 1, the RNA recovery rate of the magnetic nanoparticle was 3.6, 14, 144, and 410 times of the RNA recovery rate of the column-based RNA isolation product for the cfRNA of beta-globin, miR-21, U6, and miR-15a, respectively.

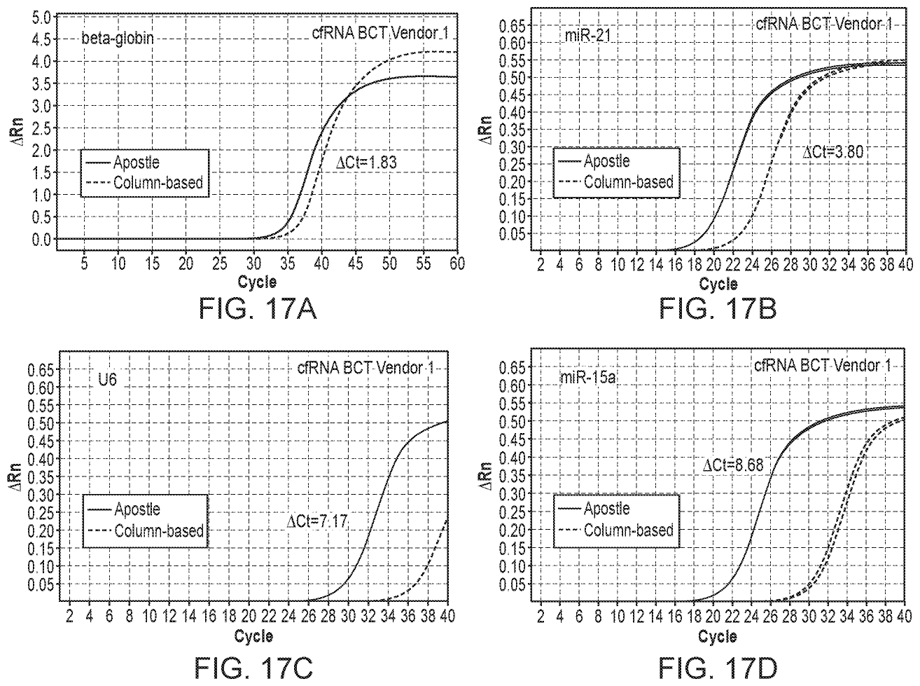

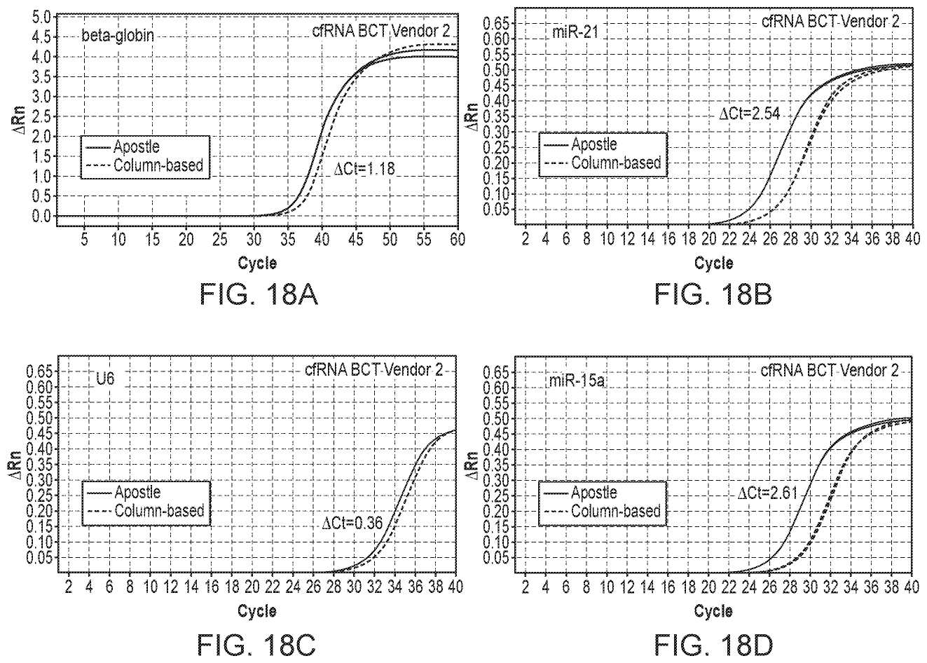

FIGS. 18A-D show that for blood collected in cfRNA BCT vendor 2, the RNA recovery rate of the magnetic nanoparticle was 2.3, 5.8, 1.3, and 6.1 times of the RNA recovery rate of the column-based RNA isolation product for the cfRNA of beta-globin, miR-21, U6, and miR-15a, respectively.

These results indicate that the magnetic nanoparticle is more efficient than the column-based RNA isolation product in isolating endogenous RNA, including microRNA, from blood collected in various blood collection tubes, especially from blood collected in the specialized BCTs that prevent RNA degradation during storage.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A magnetic nanoparticle comprising a single magnetic core and an outer shell, wherein the outer shell covers the magnetic core.

2. The magnetic nanoparticle of claim 1, wherein the magnetic particle has a maximum diameter of 100 nm to 1000 nm.

3. The magnetic nanoparticle of claim 2, wherein the magnetic particle has a maximum diameter of 300 nm to 700 nm.

4. The magnetic nanoparticle of claim 3, wherein the magnetic particle has a maximum diameter of 400 nm to 600 nm.

5. The magnetic nanoparticle of any of the preceding claims, wherein the magnetic core is composed of metal oxide.

6. The magnetic nanoparticle of claim 5, wherein the metal oxide is an iron oxide.

7. The magnetic nanoparticle of claim 6, wherein the iron oxide is Fe₃O₄.

8. The magnetic nanoparticle of claim 5, wherein the metal oxide is XFe₂O₄, wherein X is selected from the group consisting of Mn, Ca, Co, Zn, Cu, Mg, Ba, Ni, and Cr.

9. The magnetic nanoparticle of claim 1, wherein the magnetic core has a diameter of 100 nm to 800 nm.

10. The magnetic nanoparticle of claim 9, wherein the magnetic core has a diameter of 200 nm to 600 nm.

11. The magnetic nanoparticle of claim 10, wherein the magnetic core has a diameter of 300 nm to 400 nm.

12. The magnetic nanoparticle of any of the preceding claims, wherein the outer shell comprises silicon dioxide or titanium dioxide.

13. The magnetic nanoparticle of claim 12, wherein the silicon dioxide or titanium dioxide is amorphous.

14. The magnetic nanoparticle of claim 12, wherein the silicon dioxide or titanium dioxide is in crystallized form.

15. The magnetic nanoparticle of any of the preceding claims, wherein the outer shell comprises polymer.

16. The magnetic nanoparticle of claim 15, wherein the polymer is selected from the group consisting of: polyethylene glycol, polyacrylic acid, polyacrylamide, polyvinyl alcohol, poly-methyl methacrylate, polystyrene, poly-4-vinylphenol, polyester, polyimide, polyethylene, polypropylene, polyethylene vinyl acetate, polyacrylates, and polysaccharide.

17. The magnetic nanoparticle of any of the preceding claims, wherein the outer shell comprises mesoporous structure.

18. The magnetic nanoparticle of claim 17, wherein the mesoporous structure has an average surface pore diameter of 1 nm to 30 nm.

19. The magnetic nanoparticle of claim 18, wherein the mesoporous structure has an average surface pore diameter of 1 nm to 10 nm.

20. The magnetic nanoparticle of claim 18, wherein the mesoporous structure has an average surface pore diameter of 10 nm to 20 nm.

21. The magnetic nanoparticle of claim 18, wherein the mesoporous structure has an average surface pore diameter of 20 nm to 30 nm.

22. The magnetic nanoparticle of any of the preceding claims, further comprising a functional group on the surface of the outer shell.

23. The magnetic nanoparticle of claim 22, wherein the functional group is selected from the group consisting of: carboxyl, hydroxyl, epoxy, carbonyl, aldehyde, amine, maleimide, N-hydroxysuccinimide, carbodiimide, anhydride, hydrazide, and biotin.

24. The magnetic nanoparticle of any of the preceding claims, further comprising a polynucleotide, a polysaccharide, a polypeptide, a protein, an aptamer, or an ion.

25. The magnetic nanoparticle of claim 24, wherein the polynucleotide has a length of 10 to 100 bases.

26. The magnetic nanoparticle of claim 24 or 25, wherein the polynucleotide hybridizes specifically to a DNA or an RNA target.

27. The magnetic nanoparticle of any one of claims 24 to 26, wherein the polynucleotide is polydT.

28. The magnetic nanoparticle of claim 24 or 25, wherein the polynucleotide binds to a protein target.

29. The magnetic nanoparticle of claim 24, wherein the protein is an antibody.

30. The magnetic nanoparticle of claim 24, wherein the protein is Protein A, Protein G, Protein A/G, or Protein L.

31. The magnetic nanoparticle of claim 24, wherein the protein is streptavidin, avidin, or NeutrAvidin.

32. The magnetic nanoparticle of any of the preceding claims, where the magnetic nanoparticle has a positive surface charge.

33. The magnetic nanoparticle of any one of claims 1 to 31, where the magnetic nanoparticle has a negative surface charge.

34. The magnetic nanoparticle of claim 32 or 33, wherein the surface charge of the magnetic nanoparticle changes according to pH of a solution.

35. A composition comprising a plurality of magnetic nanoparticles and optionally an aqueous solution, wherein each magnetic nanoparticle has a structure of any of the preceding claims.

36. The composition of claim 35, wherein at least 40% of the magnetic particles have the same maximum diameter.

37. The composition of claim 36, wherein at least 60% of the magnetic particles have the same maximum diameter.

38. The composition of claim 37, wherein at least 80% of the magnetic particles have the same maximum diameter.

39. A kit for isolating a biological target comprising the composition of any one of claims 35 to 38 and optionally a buffer or a combination of buffers.

40. The kit of claim 39, further comprising a chaotropic agent.

41. A method of enriching one or more biological target from a biological medium, comprising:
a) providing a sample of biological medium containing one or more biological target;
b) adding to the sample a composition comprising dispersed magnetic nanoparticles capable of binding the biological target, wherein each magnetic nanoparticle has a structure of any one of claims 1 to 34, under conditions that permit a complex to form between the magnetic nanoparticle and the biological target;
c) separating the complex from the biological medium by application of an external magnetic field; and
d) recovering the biological target from the magnetic nanoparticles.

42. The method of claim 41, further comprising:
pretreating the sample of biological medium to effect the release of the biological target.

43. The method of claim 41 or 42, further comprising:
pretreating the sample of biological medium to remove contaminants.

44. The method of any one of claims 41 to 43, further comprising:
contacting the sample with a molecular probe, the molecular probe comprising a moiety with high affinity for a molecule on the magnetic nanoparticle, wherein the molecular probe binds specifically to the biological target.

45. The method of any one of claims 41 to 44, wherein the biological target is selected from the group consisting of a nucleic acid, a peptide, a protein, a carbohydrate, a lipid, a cell, and an exosome.

46. The method of claim 45, wherein the biological target is a nucleic acid.

47. The method of claim 46, wherein the nucleic acid is circulating free DNA (cfDNA) or circulating free RNA (cfRNA).

48. The method of claim 45, wherein the biological target is a cell.

49. The method of claim 48, wherein the cell is a circulating tumor cell (CTC).

50. The method of claim 41 or 42, wherein the biological medium is a body fluid.

51. The method of claim 50, wherein the body fluid is blood, serum, plasma, saliva, cerebrospinal fluid, urine, semen, or ascites.

52. A method of enriching circulating free DNA (cfDNA) from a body fluid, comprising:
a) providing a sample of body fluid containing the cfDNA;
b) adding to the sample a solution of dispersed magnetic nanoparticles capable of binding the cfDNA, wherein each magnetic nanoparticle has a structure of any one of claims 1 to 34, under conditions that permit a complex to form between the magnetic nanoparticle and the cfDNA;
c) separating the complex from the body fluid by application of an external magnetic field; and
d) recovering the cfDNA from the magnetic nanoparticles.

53. The method of claim 52, wherein a) further comprises:
pretreating the body fluid to remove cells.

54. The method of claim 52 or 53, wherein a) further comprises:
pretreating the body fluid to remove proteins.

55. The method of any one of claims 52 to 54, wherein c) further comprises:
washing the complex to remove contaminants.

56. The method of any one of claims 52 to 55, further comprising:
e) sequencing the entirety or a portion of the enriched cfDNA.

57. The method of any one of claims 52 to 56, wherein the cfDNA is less than 100 bp.

58. The method of any one of claims 52 to 56, wherein the cfDNA is a single-stranded DNA (ssDNA).

59. The method of any one of claims 52 to 58, wherein the body fluid is from a patient with, or suspected of having, cancer.

60. The method of any one of claims 52 to 58, wherein the body fluid is from a patient with, or suspected of having, an infectious disease.

61. The method of any one of claims 52 to 58, wherein the body fluid is from a pregnant woman.

62. A method of enriching circulating free RNA (cfRNA) from a body fluid, comprising:
a) providing a sample of body fluid containing the cfRNA;
b) adding to the sample a solution of dispersed magnetic nanoparticles capable of binding the cfRNA, wherein each magnetic nanoparticle has a structure of any one of claims 1 to 34, under conditions that permit a complex to form between the magnetic nanoparticle and the cfRNA;
c) separating the complex from the body fluid by application of an external magnetic field; and
d) recovering the cfRNA from the magnetic nanoparticles.

63. The method of claim 62, wherein a) further comprises:
pretreating the body fluid to remove cells.

64. The method of claim 62 or 63, wherein a) further comprises:
pretreating the body fluid to remove proteins.

65. The method of any one of claims 62 to 64, wherein c) further comprises:
washing the complex to remove contaminants.

66. The method of any one of claims 62 to 65, further comprising:
e) sequencing the entirety or a portion of the enriched cfRNA.

67. The method of any one of claims 62 to 66, wherein the cfRNA is less than 100 nt.

68. The method of any one of claims 62 to 67, wherein the cfRNA is a miRNA.

69. The method of any one of claims 62 to 68, wherein the body fluid is from a patient with, or suspected of having, cancer.

70. The method of any one of claims 62 to 68, wherein the body fluid is from a patient with, or suspected of having, an infectious disease.

71. The method of any one of claims 62 to 68, wherein the body fluid is from a pregnant woman.

72. A method of enriching circulating tumor cell (CTC) from a body fluid, comprising:
a) providing a sample of body fluid containing the circulating tumor cell;
b) adding to the sample a solution of dispersed magnetic nanoparticles capable of binding the circulating tumor cell, wherein each magnetic nanoparticle has a structure of any one of claims 1 to 34, under conditions that permit a complex to form between the magnetic nanoparticle and the circulating tumor cell; and
c) separating the complex from the body fluid by application of an external magnetic field.

73. The method of claim 72, wherein a) further comprises:
pretreating the body fluid to enrich cells.

74. The method of claim 72 or 73, wherein c) further comprises:
washing the complex to remove contaminants.

75. The method of any one of claims 72 to 74, further comprising:
recovering the circulating tumor cell from the magnetic nanoparticles.

76. The method of any one of claims 72 to 75, further comprising:
analyzing the circulating tumor cell.

77. The method of claim 76, wherein the analyzing the circulating tumor cell is analyzing the size and shape of the circulating tumor cell, analyzing the surface biomarker of the circulating tumor cell, or sequencing the DNA/RNA of the circulating tumor cell.

78. The method of any one of claims 72 to 77, wherein the body fluid is from a patient with, or suspected of having, cancer.

79. A method of preparing a magnetic nanoparticle, comprising:
a) making a dispersion comprising a metal salt, an organic solvent, and a capping reagent;
b) heating the dispersion;
c) isolating magnetic cores from the dispersion;
d) adding a silicon or titanium organic compound to the magnetic cores;
e) hydrolyzing at least some of the silicon or titanium organic compound; and
f) crosslinking the hydrolyzed silicon or titanium organic compound on the surface of the magnetic cores.

80. The method of claim 79, wherein the metal salt is an iron salt.

81. The method of claim 79, wherein the metal salt is an iron salt and a salt of a second metal.

82. The method of claim 81, wherein the second metal is selected from the group consisting of Mn, Ca, Co, Zn, Cu, Mg, Ba, Ni, and Cr.

83. The method of any one of claims 79 to 82, wherein the heating comprises heating the dispersion to 180-240° C. for 4-80 hours.

84. The method of any one of claims 79 to 83, wherein the isolating comprises cooling, washing, and drying.

85. The method of any one of claims 79 to 84, wherein the dispersion further comprises a first surfactant.

86. The method of any one of claims 79 to 85, wherein before adding the silicon or titanium organic compound to the magnetic cores, d) further comprises dispersing the magnetic cores in solution comprising a second surfactant, wherein the second surfactant self-assembles on the magnetic core.

87. The method of claim 86, further comprising:
g) removing the self-assembled surfactant by ion exchange.

88. The method of claim 86 or 87, wherein the magnetic nanoparticle is mesoporous.
